(19) United States
(12) Patent Application Publication
(10) Pub. No.: US 2019/0021676 A1
(43) Pub. Date: Jan. 24, 2019

(54) INFORMATION PROCESSING APPARATUS

(71) Applicants: Michinari SHINOHARA, Kanagawa (JP); Yutaka YAGIURA, Kanagawa (JP); Noriyuki TOMITA, Ishikawa (JP); Daisuke SAKAI, Tokyo (JP)

(72) Inventors: Michinari SHINOHARA, Kanagawa (JP); Yutaka YAGIURA, Kanagawa (JP); Noriyuki TOMITA, Ishikawa (JP); Daisuke SAKAI, Tokyo (JP)

(73) Assignee: Ricoh Company, Ltd., Tokyo (JP)

(21) Appl. No.: 16/113,501

(22) Filed: Aug. 27, 2018

Related U.S. Application Data
(63) Continuation of application No. PCT/JP2018/009772, filed on Mar. 13, 2018.

(30) Foreign Application Priority Data
Mar. 17, 2017 (JP) 2017-053647
Oct. 24, 2017 (JP) 2017-205670

(51) Int. Cl. A61B 5/00; A61B 5/04; A61B 5/0476

(52) U.S. Cl. CPC A61B 5/7235; A61B 5/7435; A61B 5/0476; A61B 5/04012; A61B 5/04008

(57) ABSTRACT

An information processing apparatus includes a display control unit. The display control unit is configured to execute control to display a plurality of partial waveform data in parallel in a time axis direction of the plurality of partial waveform data, the plurality of partial waveform data each representing temporal change of one or more biological signals and each respectively representing biological waveform data corresponding to a respective partial time period, spots from among the biological waveform data being specifiable, the display control unit being further configured to execute control to display the spots of the plurality of partial waveform data, with emphasis, upon being specified.

### CROSS-REFERENCE TO RELATED APPLICATIONS

The present application is a continuation application of International Application No. PCT/JP2018/009772, filed Mar. 13, 2018, which claims priority to Japanese Patent Application No. 2017-053647, filed Mar. 17, 2017, and Japanese Patent Application No. 2017-205670, filed Oct. 24, 2017. The contents of these applications are incorporated herein by reference in their entirety.

### BACKGROUND OF THE INVENTION

### 1. Field of the Invention

The present invention relates to an information processing apparatus.

### 2. Description of the Related Art

A technique for estimating an in vivo signal source, based on a measured biological signal of a subject, and displaying the in vivo signal source superimposed on a tomographic image has been known (see, for example, Japanese Laid-open Patent Publication No. 2000-005133).

In this technique, with a magnetoencephalograph or an electroencephalograph for measurement of neural activity of a brain, for example, a signal source is displayed superimposed on a tomographic image, based on a specific waveform part (a singular point, for example) of a waveform of a biological signal, and a doctor or the like identifies a position to be removed by surgery (for example, a part being a cause of epilepsy).

However, because the conventional device displays the waveform of the biological signal as is, an operator is unable to compare, on a single screen, plural singular points or the like included in the biological signal. The conventional device has a problem that identification of a target part being a cause of a case is not sufficient, because depending on a waveform part, the waveform part may need to be excluded before signal source estimation.

### SUMMARY OF THE INVENTION

According to one aspect of the present invention, an information processing apparatus includes a display control unit. The display control unit is configured to execute control to display a plurality of partial waveform data in parallel in a time axis direction of the plurality of partial waveform data, the plurality of partial waveform data each representing temporal change of one or more biological signals and each respectively representing biological waveform data corresponding to a respective partial time period, spots from among the biological waveform data being specifiable, the display control unit being further configured to execute control to display the spots of the plurality of partial waveform data, with emphasis, upon being specified.

### BRIEF DESCRIPTION OF THE DRAWINGS

FIG. 1 is a schematic diagram illustrating an example of a biological signal measurement system according to a first embodiment;

FIG. 2 is a diagram illustrating an example of a hardware configuration of an information processing apparatus;

FIG. 3 is a diagram illustrating an example of functional blocks of the information processing apparatus;

FIG. 4 is a diagram illustrating an example of a start screen displayed on the information processing apparatus;

FIG. 5 is a diagram illustrating an example of a measurement recording screen;

FIG. 6 is an enlarged view of a left side area of the measurement recording screen;

FIG. 7 is an enlarged view of a right side area of the measurement recording screen;

FIG. 8 is a diagram illustrating a screen immediately after annotation information is input;

FIG. 9 is a diagram illustrating an updated annotation list;

FIG. 10 is a flow chart illustrating an example of information display processing by the information processing apparatus at the time of measurement recording;

FIG. 11 is a diagram illustrating an example of an analysis screen;

FIG. 12 is an enlarged view of a left side area of the analysis screen;

FIG. 13 is an enlarged view of a right side area of the analysis screen;

FIG. 14 is a diagram illustrating a screen immediately after a specific annotation line is selected on the analysis screen;

FIG. 15 is an enlarged view of a left side area of the screen in FIG. 14;

FIG. 16 is an enlarged view of a right side area of the screen in FIG. 14;

FIG. 17 is a flow chart illustrating an example of information display processing of the information processing apparatus at the time of analysis;

FIG. 18 is a diagram illustrating an example of a screen displayed when a merging button is pressed down;

FIG. 19 is a diagram for explanation of an example of tomographic images in a display window;

FIG. 20 is a diagram for explanation of switch-over of display of slice images in the display window;

FIG. 21 is a diagram illustrating a modification of the screen displayed when the merging button is pressed down;

FIG. 22 is a diagram illustrating an example of a screen displayed when a merging button is pressed down in a second embodiment;

FIG. 23 is a flow chart illustrating an example of operation at the time of measurement recording by an information processing apparatus according to a third embodiment;

FIG. 24 is a flow chart illustrating an example of operation at the time of analysis by the information processing apparatus according to the third embodiment;

FIG. 25 is a diagram illustrating a left side area of an example of an analysis screen according to the third embodiment;

FIG. 26 is a diagram illustrating a modification of the analysis screen according to the third embodiment;

FIGS. 27A to 27E are diagrams for explanation of display methods for distinction between signal waveforms for different pieces of range information, according to the third embodiment;

FIG. 28 is a schematic diagram illustrating that each of plural measurement files is managed in association with analysis files, in the third embodiment;

FIG. 29 is a diagram illustrating correspondence between analysis files of the same analyst and measurement files when these analysis files are extracted, in the third embodiment;

FIG. 30 is a flow chart illustrating an example of information display processing by an information processing apparatus at the time of measurement recording, according to a fourth embodiment;

FIG. 31 is a diagram illustrating an example of a measurement recording result screen called from a measurement recording screen, according to the fourth embodiment;

FIG. 32 is a diagram illustrating a modification of the measurement recording result screen according to the fourth embodiment;

FIG. 33 is a diagram illustrating another modification of the measurement recording result screen according to the fourth embodiment;

FIG. 34 is a diagram illustrating still another modification of the measurement recording result screen according to the fourth embodiment;

FIG. 35 is a diagram illustrating yet another modification of the measurement recording result screen according to the fourth embodiment;

FIG. 36 is a diagram illustrating another modification of the measurement recording result screen according to the fourth embodiment;

FIG. 37 is a diagram illustrating, as a modification of the fourth embodiment, a display example for an annotation;

FIG. 38 is a diagram illustrating, as another modification of the fourth embodiment, a display example for waveforms;

FIG. 39 is a flow chart illustrating, as a fifth embodiment, an example of operation of switch-over from a measurement recording result screen to an analysis screen; and

FIG. 40 is a diagram illustrating, as a sixth embodiment, an example of a biological image.

The accompanying drawings are intended to depict exemplary embodiments of the present invention and should not be interpreted to limit the scope thereof. Identical or similar reference numerals designate identical or similar components throughout the various drawings.

### DETAILED DESCRIPTION OF THE PREFERRED EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

Hereinafter, while reference is made to the appended drawings, embodiments of an information processing apparatus, an information processing method, a computer-readable medium, and a biological signal measurement system, according to the present invention, will be described in detail.

An embodiment has an object to provide an information processing apparatus which enable improvement in reliability of identification of a target part being a cause of a case.

### First Embodiment

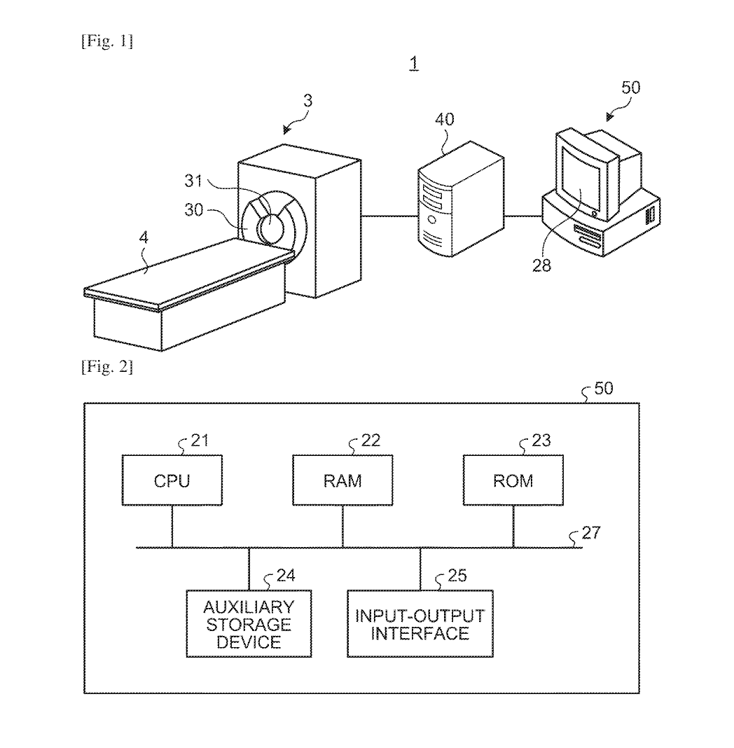

FIG. 1 is a schematic diagram illustrating an example of a biological signal measurement system according to a first embodiment. The biological signal measurement system measures plural types of biological signals of a person to be measured (subject), for example, magnetoencephalography (MEG) signals and electroencephalography (EEG) signals, and displays these biological signals. The biological signals to be measured are not limited to the magnetoencephalography signals and the electroencephalography signals, and may be, for example, electrical signals generated according to cardiac activity (electrical signals expressible as an electrocardiogram). As illustrated in FIG. 1, a biological signal measurement system 1 includes: a measurement apparatus 3 that measures one or more biological signals of a subject; a server 40 that records therein the one or more biological signals measured by the measurement apparatus 3; and an information processing apparatus 50 that analyzes the one or more biological signals recorded in the server 40. A part or all of functions that the server 40 has may be incorporated in the information processing apparatus 50.

In the example of FIG. 1, the subject lies on his back on a measurement table 4, in a state where electrodes (or sensors) for electroencephalographic measurement are attached to his head, and the subject puts his head in a hollow 31 of a Dewar 30 of the measurement apparatus 3. The Dewar 30 is a holding container that uses liquid helium and that is under a cryogenic environment, and multiple magnetic sensors for magnetoencephalographic measurement are arranged inside the hollow 31 of the Dewar 30. The measurement apparatus 3 collects signals acquired by the respective measurement methods, that is, electroencephalography signals from the electrodes and magnetoencephalography signals from the magnetic sensors, and the measurement apparatus 3 outputs data including the collected electroencephalography signals and magnetoencephalography signals (which may be referred to as “measurement data” in the following description) to the server 40. The measurement data recorded in the server 40 are read out by the information processing apparatus 50 and are displayed on a display screen of a display device 28, and assignment of annotations and analysis, which will be described later, are performed. Generally, the Dewar 30 having the built-in magnetic sensors, and the measurement table 4 are arranged in a magnetically shielded room, but for convenience of illustration, illustration of the magnetically shielded room is omitted.

The electroencephalography signals and the magnetoencephalography signals are an example of the “biological signals”. An electroencephalography signal represents electrical activity of a neuron (flow of ionic charge caused at a dendrite of the neuron upon synaptic transmission), as voltage value between electrodes. A magnetoencephalography signal represents minute magnetic field fluctuation caused by electrical activity of the brain. The cerebral magnetic field is detected by a highly sensitive superconducting quantum interference device (SQUID) sensor.

The information processing apparatus 50 displays waveforms of the magnetoencephalography signals from the plural magnetic sensors and waveforms of the electroencephalography signals from the plural electrodes, which are represented by the measurement data, in synchronization with each other on the same time axis. Data of the signal waveforms of the electroencephalography signals and the data of the signal waveforms of the magnetoencephalography signals disclosed herein are an example of “biological data” representing temporal changes of the biological signals.

FIG. 2 is a diagram illustrating an example of a hardware configuration of the information processing apparatus 50. The information processing apparatus 50 has a CPU (central processing unit: processor) 21, a random access memory (RAM) 22, a read only memory (ROM) 23, an auxiliary storage device 24, and an input-output interface 25; and these are connected to one another via a bus 27.

The CPU 21 controls the overall operation of the information processing apparatus 50, and executes various types of information processing. For example, the CPU 21 expands and executes, on the RAM 22, a control program stored in the ROM 23 or the auxiliary storage device 24, and executes display control processing for, for example, a measurement recording screen and an analysis screen, which will be described later. The RAM 22 is used as a work area of the CPU 21. As the RAM 22, a partial area of a nonvolatile RAM storing therein main control parameters and information may be used. The ROM 23 stores therein a basic input and output program, or the like. The auxiliary storage device 24 is a storage device, such as a solid state drive (SSD) or a hard disk drive (HDD), and stores therein, for example: a control program for control of the operation of the information processing apparatus 50; and various data, files, and the like necessary for the operation of the information processing apparatus 50. The control program may be stored in the ROM 23.

The input-output interface 25 has a user interface for each of peripheral devices (for example, a touch panel, a keyboard, a mouse, the display device 28, operation buttons, a printer, and the like), and a communication interface that transmits and receives information from the sensors and the electrodes by communicating with the server 40. The touch panel, the keyboard, the mouse, and the operation buttons are input devices, and notify the CPU 21 of input signals (operation signals) via the input-output interface 25. In this embodiment, description will be made mainly with an example where a mouse is used. The display device 28 is a display device (display) that displays thereon various types of information; and the display device 28 receives, via the input-output interface 25, display information (including the measurement recording screen and the analysis screen) processed by the CPU 21 based on operation signals and the like from the input device, and displays the received display information on a screen. Based on instructions from the CPU 21, the printer records print data output from the input-output interface 25 on a recording medium, such as a sheet of paper.

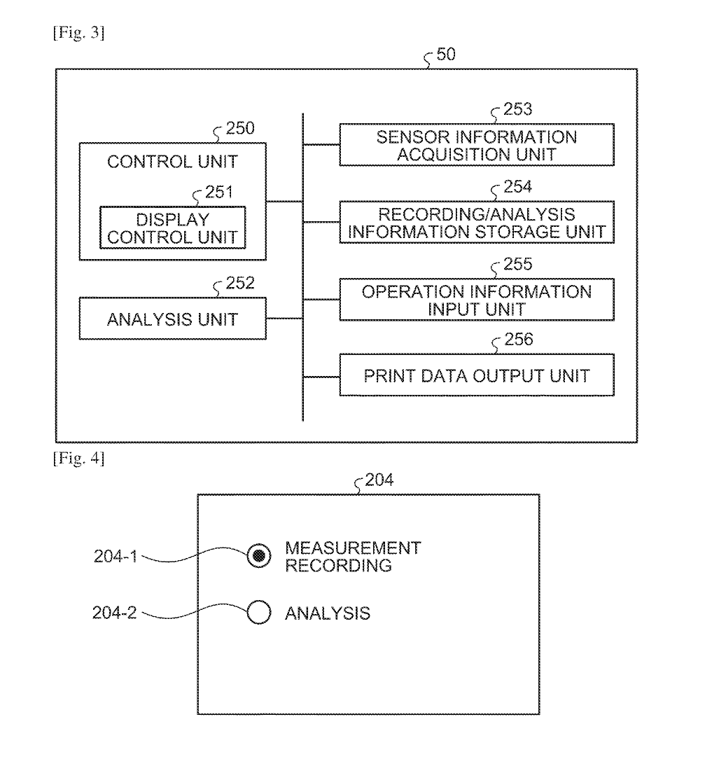

FIG. 3 is a diagram illustrating an example of functional blocks of the information processing apparatus 50. The information processing apparatus 50 has a control unit 250, an analysis unit 252, a sensor information acquisition unit 253, a recording/analysis information storage unit 254, an operation information input unit 255, and a print data output unit 256. The control unit 250 includes a display control unit 251 that controls the screen display by the information processing apparatus 50.

The sensor information acquisition unit 253 acquires sensor information from the server 40. The sensor information refers to the measurement data at the time of measurement recording by use of the measurement apparatus 3, and refers to the measurement data and information on annotations added to the respective signal waveforms at the time of analysis in the information processing apparatus 50. The annotations will be described in detail later. According to operation on the input device, the operation information input unit 255 inputs annotations and operation signals. The analysis unit 252 executes analysis of signal waveforms, analysis of singular points of amplitude, analysis of cerebral magnetic fields including directions of current dipoles, and the like. “Signal source estimation” described later is executed in these analyses. The recording/analysis information storage unit 254 stores the sensor information and results of the analyses to a specified destination. If annotations are assigned at the time of measurement recording, the annotations are also stored. In this example, the specified destination is the server 40, but the specified destination may be the auxiliary storage device 24, or the like. If the specified destination is the auxiliary storage device 24, the sensor information acquisition unit 253 may be modified to directly receive the biological signals from the measurement apparatus 3 without the biological signals going through the server 40. The print data output unit 256 outputs the print data to the printer. The output to the printer, which is a peripheral device, is just an example, and screen display data, the print data, and the like may be output to another terminal via a network, for example, and that terminal may execute display, printing, and the like.

The control unit 250 receives and outputs information from and to each functional unit, and controls the screen display on the display device 28 according to results of processing based on the information. Specifically, the display control unit 251 controls the screen display on the display device 28 via the input-output interface 25. In this embodiment, the control unit 250 mainly controls the screen display for the sensor information at the time of measurement recording and at the time of analysis.

The control unit 250 and the analysis unit 252 are implemented by the CPU 21 in FIG. 2 expanding and executing, on the RAM 22, the control program stored in the ROM 23 or the like. Not being limited to this configuration, for example, at least a part of the control unit 250 and analysis unit 252 may be implemented by a dedicated hardware circuit (semiconductor integrated circuit, or the like). The sensor information acquisition unit 253, the operation information input unit 255, and the print data output unit 245 are implemented mainly in the input-output interface 25, in this example. Functions of the recording/analysis information storage unit 254 are implemented mainly in the auxiliary storage device 24 and the input-output interface 25, in this example.

Hereinafter, by description of display modes of the measurement recording screen and the analysis screen by use of the drawings, control of the display screen executed by the control unit 250 (mainly the display control unit 251) will be described. As to phrases, such as “to be displayed” and “being displayed”, in the following description, a means that executes the display is the display control unit 251. Further, operation information, such as “selection”, “depression”, and “input”, is received from the operation information input unit 255.

FIG. 4 is a diagram illustrating an example of a start screen 204 displayed on the information processing apparatus 50. On the start screen 204, “Measurement Recording” and “Analysis” selection buttons are displayed. In electroencephalographic measurement and magnetoencephalographic measurement, measurement recording of data and analysis of data are often executed by different persons. For example, when a “Measurement Recording” button 204-1 is selected by a measurement technician (measurer), data measured by the measurement apparatus 3 are sequentially stored in the server 40, and are read out and displayed by the information processing apparatus 50. When an “Analysis” button 204-2 is selected by a doctor after the measurement recording is ended, the data recorded by the measurement recording are read out and analyzed.

### Operation in Measurement Recording

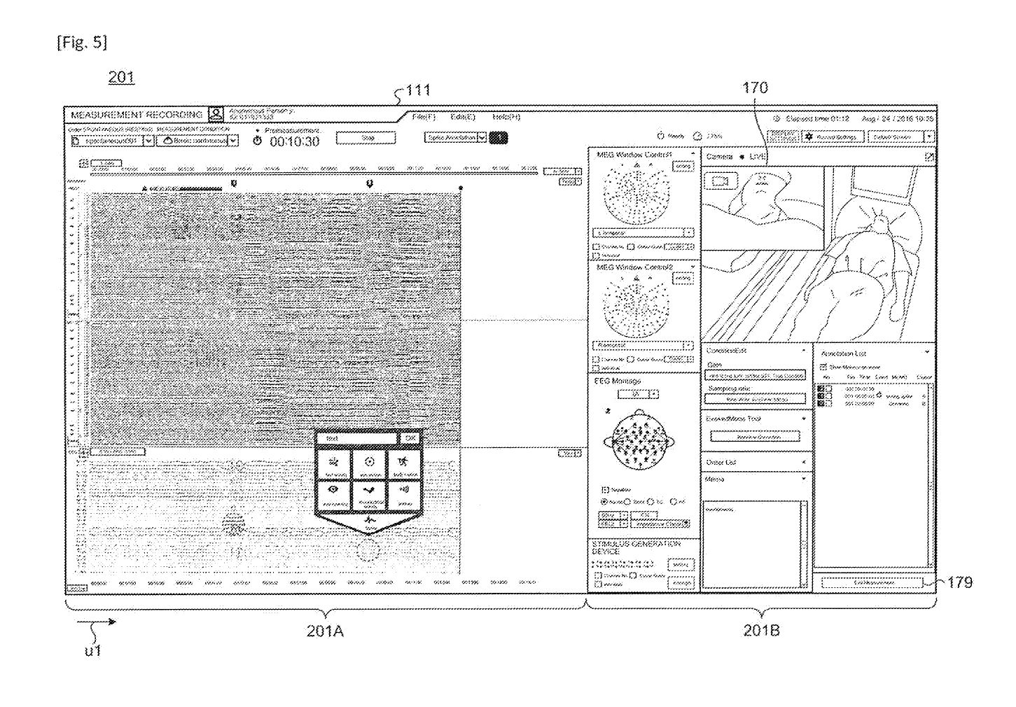

FIG. 5 is a diagram illustrating an example of the measurement recording screen. A measurement recording screen (which is alternatively simply referred to as “screen”) 201 illustrated in FIG. 5 displays thereon that the screen 201 is a “Measurement Recording” screen in a tab 111 on the screen 201. The measurement recording screen 201 has an area 201A where signal waveforms of biological signals are displayed, and an area 201B where monitoring information other than the signal waveforms is displayed. The area 201A where the waveforms are displayed is arranged at a left side on the screen 201 as viewed from the measurer, and the area 201B where the monitoring information other than the signal waveforms is displayed is arranged at a right side on the screen 201 as viewed from the measurer. No waste is caused: in eye movements of the measurer following movement of the waveforms detected in realtime and displayed horizontally rightward (in a direction u1) from a left end on the screen 201; and in movement when the mouse is moved from the area 201A at the left side on the screen 201 to the area 201B at the right side thereon; and work efficiency is thus improved. “Horizontally” referred to above and hereinafter refers to being on an arbitrary line parallel to the direction u1 on the screen 201. Further, in this specification, when the diagrams of the whole screen are referred to, “left”, “right”, “up”, and “down” are used in the description as appropriate, and these “left”, “right”, “up”, and “down” refer to “left”, “right”, “up”, and “down”, respectively, in a case where the diagrams of the whole screen are viewed with the direction u1 appended thereto being made horizontal.

In the area 201B, a monitoring window 170 for check of the state of the person to be measured during the measurement is displayed. By display of live video of the person to be measured during the measurement, as described later, reliability of check and determination of the signal waveforms is able to be increased. FIG. 5 illustrates a display mode in the case where the whole measurement recording screen 201 is displayed on the display screen of the single display device 28 (see FIG. 1), but the area 201A and the area 201B may be separately displayed by being divided to two or more display devices.

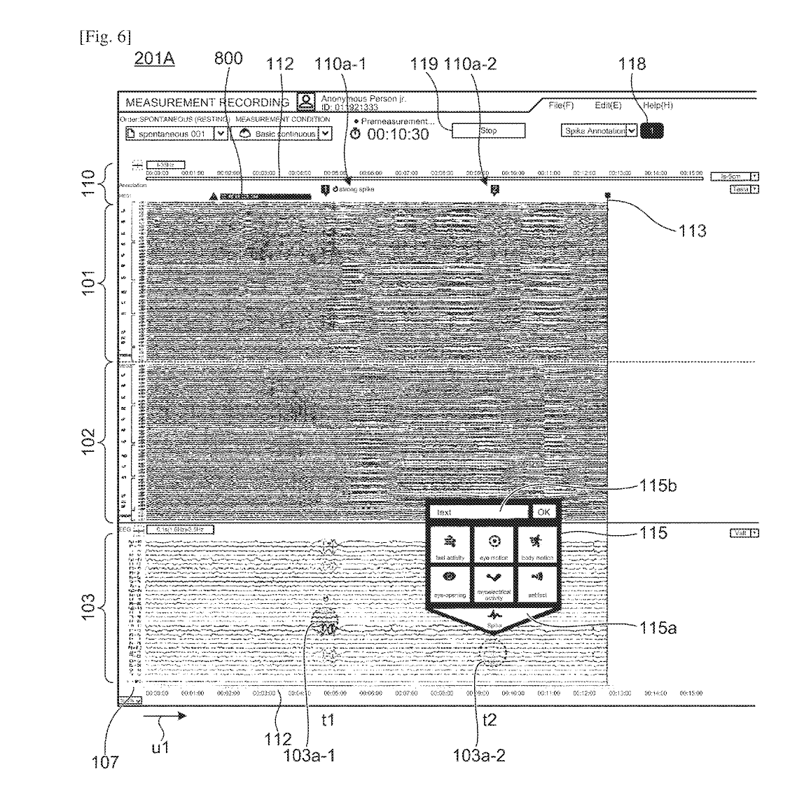

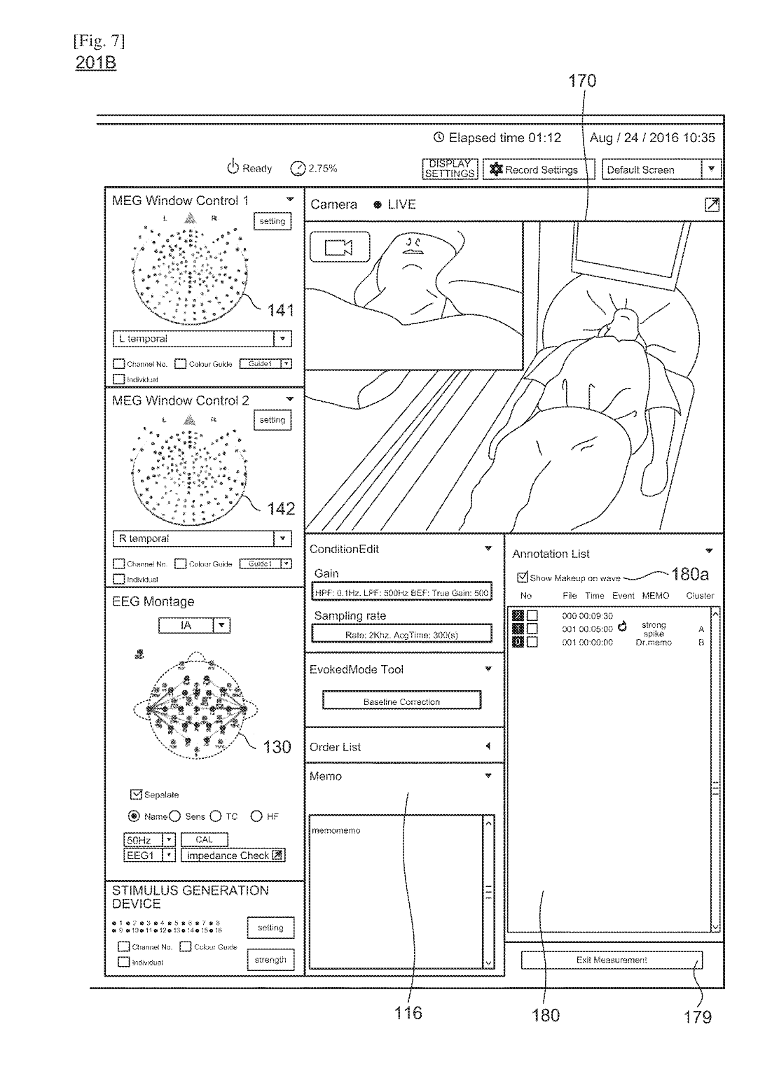

FIG. 6 is an enlarged view of the area 201A illustrated in FIG. 5. The area 201A has a display area 110 where time information of signal detection is displayed, and display areas 101 to 103 where plural sets of signal waveforms based on signal detection are parallelly displayed.

The time information displayed in the display area 110 is a time line including time display assigned with times (numbers) along a time axis 112 in the example of FIG. 6, but the time information may be just a zonal axis without display of the times (numbers), or only the times (numbers) may be displayed without provision of the axis. Further, in addition to the display area 110, the same time axis 112 may be displayed below the display area 103, as illustrated in FIG. 6.

In the display areas 101 to 103, plural sets of signal waveforms are parallelly displayed, horizontally. As each set of signal waveforms, only signal waveforms from the same sensor group may be displayed, or signal waveforms from different sensor groups may be displayed together. Further, signal waveforms from an electrode group may be displayed, alone, or together with other signal waveforms. Among these, one or more types of signal waveforms are displayed. The same sensor group may be classified by, for example, the site measured. In this example, classification is made into: waveforms of magnetoencephalography signals acquired from a magnetic sensor group corresponding to a right side of the head of the person to be measured; waveforms of magnetoencephalography signals acquired from a magnetic sensor group corresponding to a left side of the head of the person to be measured; and waveforms of electroencephalography signals acquired from electrodes for electroencephalographic measurement on the person to be measured. The combination of the “plural sets of signal waveforms” is not limited to the above combination. For example, any of parts, such as the parietal region, the frontal lobe, and the temporal lobe, may be selected, and signal waveforms acquired from sensors for each of the selected parts may be selectively displayed. A method of selecting the signal waveforms will be described in detail later.

In FIG. 6, a set of the waveforms of the plural magnetoencephalography signals acquired from the right side of the head of the person to be measured is displayed in the display area 101, a set of the waveforms of the plural magnetoencephalography signals acquired from the left side of the head of the person to be measured is displayed in the display area 102, and a set of the waveforms of the plural electroencephalography signals is displayed in the display area 103, each horizontally and parallelly to one another. These signal waveforms are displayed in synchronization with one another on the same time axis. Correspondingly with each of these signal waveforms, a channel number 107 where that signal has been acquired, specifically, identification information of the electrodes (for example, identification information of the reference electrode and the active electrode) or an identification number of the sensor is displayed. In this embodiment, identification information of the electrodes has one-to-one correspondence to channel numbers, identification information of the sensors has one-to-one correspondence to the channel numbers; and the identification information of the electrodes, the identification information of the sensors, and the channel numbers will be used, as appropriate, in the description. Not being limited thereto, plural electrodes, or plural sensors may be associated in a group with a channel number.

When measurement is started, measurement information from each sensor and each electrode is collected, and signal waveforms are horizontally displayed in order of time of measurement, rightward (in the direction u1) from the left end in the respective display areas 101 to 103. A line 113 indicates the current time, at which the measurement is being executed, and moves rightward from a left end of the area 201A. After the signal waveforms are displayed up to a right end of the area 201A (a right end of the time axis 112), the signal waveforms gradually disappear rightward from the left end of the area 201A, signal waveforms are newly displayed rightward from the left end sequentially at the position where the signal waveforms disappeared, and the line 113 also moves rightward from the left end. In association therewith, display of time on the time axis 112 is updated according to the range of elapsed time for the newly displayed signal waveforms. The measurement recording is continued until an “Exit Measurement” button 179 is pressed.

This embodiment is characterized in that when the measurer (recorder) notices a waveform disturbance, a singular point of amplitude, or the like, on signal waveforms during recording of data, the measurer is able to mark that spot (a spot to be noted) on the signal waveforms. The position and range of the mark are able to be specified by pointer operation or click operation by use of a mouse. The spot to be noted is displayed with emphasis by: the mark being displayed on the signal waveforms in the display areas 101 to 103; and a result of that specification being displayed at a position (corresponding time position) along the time axis 112 in the display area 110. Information on the marking including the display along the time axis 112 is stored in the specified destination, together with the signal waveform data (biological data). The term, “spot to be noted”, is used above and hereinafter as a concept including, not just the signal waveform at a certain point, but also signal waveform or waveforms of a certain range.

FIG. 6 illustrates, as an example, a display mode in a case where a range has been specified, the range including a waveform disturbance (a spot to be noted) on one or more channels in the display area 103 at a time t1. As illustrated in FIG. 6, the spot to be noted is displayed with emphasis with a mark 103a-1. Further, emphasized display is executed in the display area 110, by display of an annotation 110a-1 indicating a result of the specification, at a time position corresponding to the mark 103a-1. The emphasized display may be highlighting in the mark 103a-1, or display of an annotation near the mark 103a-1. Furthermore, in FIG. 6, since there is a disturbance in the waveforms at a time t2 also, a mark 103a-2 and an annotation 110a-2 indicating a result of the specification are displayed for that spot to be noted also.

Although the mark 103a-1 and the mark 103a-2 are circular in the illustration, they may be shaped differently, for example, rectangularly. For example, if they are to be shaped circularly, the mark 103a-1 is provided by specification of a radius of the mark 103a-1 and specification, through a click operation, of a point where the central point of the mark 103a-1 is to be arranged. A predetermined value may be set as the radius of the mark 103a-1 beforehand, or the measurer may set an arbitrary value when specifying the spot to be noted. The same applies to the mark 103a-2.

Annotation refers to assignment of related information, as an annotation, to certain data. In this embodiment, when an “annotation” is stated without any particular specification of a target, that “annotation” refers to a mark or an icon for emphasized display of a spot to be noted. In FIG. 6, for example, the mark 103a-1, the mark 103a-2, the annotation 110a-1, and the annotation 110a-2 correspond to “annotations”. Hereinafter, without particular explanation, anything used for emphasized display will be described as an “annotation”.

The annotation 110a-1 added in the display area 110 at the time t1 includes, for example, an annotation identification number and information indicating an attribute of the waveforms. In this example, together with an annotation number, “1”, an icon indicating the attribute of the waveforms and text information, “strong spike”, are displayed.

When the measurer specifies another waveform spot or an area near that waveform spot at the time t2, the specified spot is displayed with emphasis with the mark 103a-2, and in association therewith, at a time position corresponding thereto in the display area 110, an annotation number, “2”, is displayed. Further, at the spot displayed with emphasis, a pop-up window 115 for selection of an attribute is displayed. The pop-up window 115 has selection buttons 115a for selection of various attributes, and an input box 115b for input of comments and additional information. On the selection buttons 115a, causes of a disturbance in waveforms, such as “fast activity”, “eye motion”, “body motion”, and “spike”, are displayed as attributes of waveforms. Since the measurer is able to check the state of the person to be measured on the monitoring window 170 in the area 201B on the screen 201 (see FIG. 5), the measurer is able to select an attribute indicating a cause of a disturbance in the waveforms appropriately. For example, when a spike is caused in waveforms, whether the spike is a spike representing a symptom of epilepsy or a spike caused by body motion (sneezing or the like) of the person to be measured is able to be determined.

Further, instead of the checking method by the use of the monitoring window 170, a sensor for detecting motion of the person to be measured may be provided and a warning display 800 as illustrated in FIG. 6 may be displayed in the display area 110 over the detected time period. For example, if the head of the person to be measured moves, and the amount of that movement exceeds a permissible amount, the warning display 800 is displayed. In the warning display 800, the sensor numbers unusable for waveform data (biological data) are displayed. Thereby, the measurer is able to determine whether the spike is a spike representing a symptom of epilepsy or a spike caused by body motion (sneezing or the like) of the person to be measured. As the warning display 800, different types of “body motion”, or “eye motion” and “body motion” may be displayed in different colors.

Further, instead of the display with the warning display 800, an attribute of the waveforms in the pop-up window 115 may be automatically input, based on, for example, an output from the sensor that detects motion of the person to be measured.

The same operation is executed at the time t1 also, and in FIG. 6, by selection of the selection button 115a for “spike” in the pop-up window 115 and input of “strong spike” in the input box 115b, the annotation 110a-1 is displayed in the display area 110. According to this display mode, when multiple waveforms are displayed in synchronization with one another, a spot to be noted in the signal waveforms is able to be easily identified on the same time axis 112 by visual recognition, and basic information on the spot to be noted is able to be understood easily.

A part or all of the annotation 110a-1, for example, at least one of the attribute icon and the text annotation, may be displayed near the mark 103a-1 on the signal waveforms in the display area 103. The addition of the annotation on the signal waveforms may obstruct the check on the shapes of the waveforms, and thus when annotations are displayed on the signal waveforms in the display areas 101 to 103, display or non-display of the annotations is desirably made selectable.

In a counter box 118, a cumulative number of spike annotations is displayed. Every time “spike” is selected, the count value in the counter box 118 is incremented, and the total number of spikes from the start of the recording to the present (the line 113) is able to be recognized at a glance.

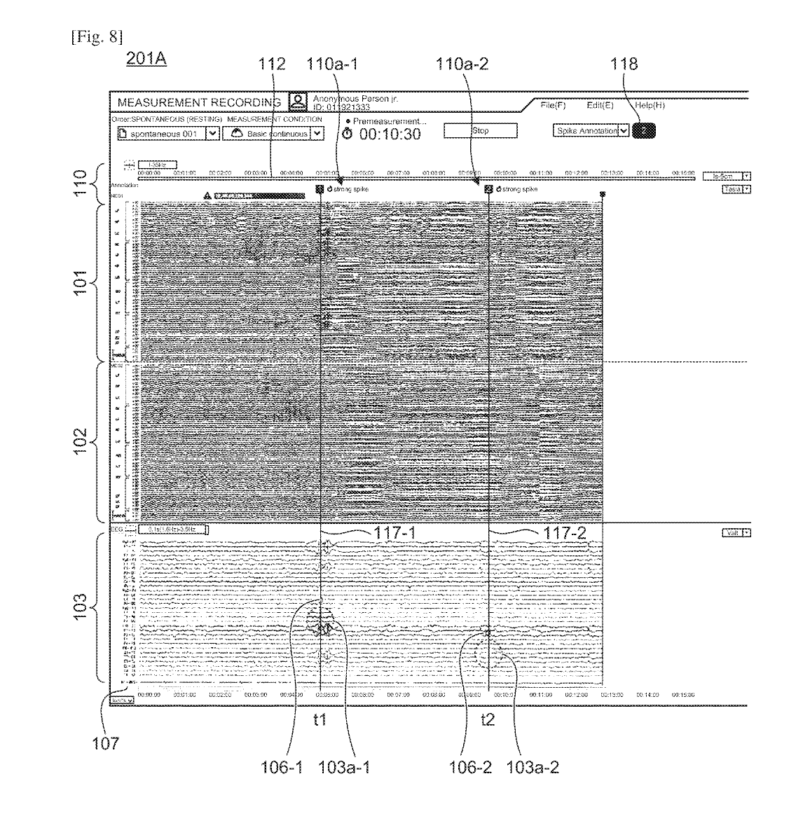

FIG. 7 is an enlarged view of the area 201B illustrated in FIG. 5, and illustrates a state at the same time (the time point of the line 113) as FIG. 6. In the monitoring window 170 in the area 201B, live video of the state of the person to be measured lying on the measurement table 4 with his head put in the measurement apparatus 3 is displayed.

In addition, in the area 201B: distribution diagrams 141, 142, and 130 (which will be described later) corresponding to the signal waveforms in the display areas 101, 102, and 103, respectively (see FIG. 6); an annotation list 180; and the like, are displayed. The annotation list 180 is a list of annotations added through marks on the signal waveforms in FIG. 6. Every time a spot to be noted on the signal waveforms is specified in the display areas 101 to 103 and an annotation is assigned thereto, information corresponding to the annotation is added to the annotation list 180 in order. The addition and display of information to and in the annotation list 180 are executed in, for example, descending order (with the newer data being displayed up), but limitation is not made to this example. The information may be displayed in ascending order, but if the information is displayed in ascending order, the information is displayed such that correspondence thereof to the annotations displayed along the time axis 112 in the display area 110 (see FIG. 6) is able to be known therefrom. Further, the display order may be changed, or the information may be sorted by item.

In the example of FIG. 7, time information corresponding to the annotation number, “1”, and information on the added annotation are listed. As the information on the annotation: the attribute icon representing “spike”; and text, “strong spike”, are recorded. Further, at a point in time when the emphasized display is executed with the mark 103a-2, the time information corresponding to the annotation number, “2”, has been listed. In the annotation list 180: the annotation number, the time information, and the information on the annotation may be displayed in a set; only the information on the annotation may be displayed; or the information on the annotation and the annotation number in a pair, or the information on the annotation and the time information in a pair, may be displayed.

Further, a selection box 180a for display/non-display is arranged near the annotation list 180. When non-display is selected in the selection box 180a, the annotations other than the marks for emphasized display on the signal waveforms are not displayed in the display areas 101 to 103, but the display of the annotations along the time axis 112 in the display area 110 is maintained. Thereby, without obstruction of the visibility of the signal waveforms, information on the annotations is able to be made recognizable.

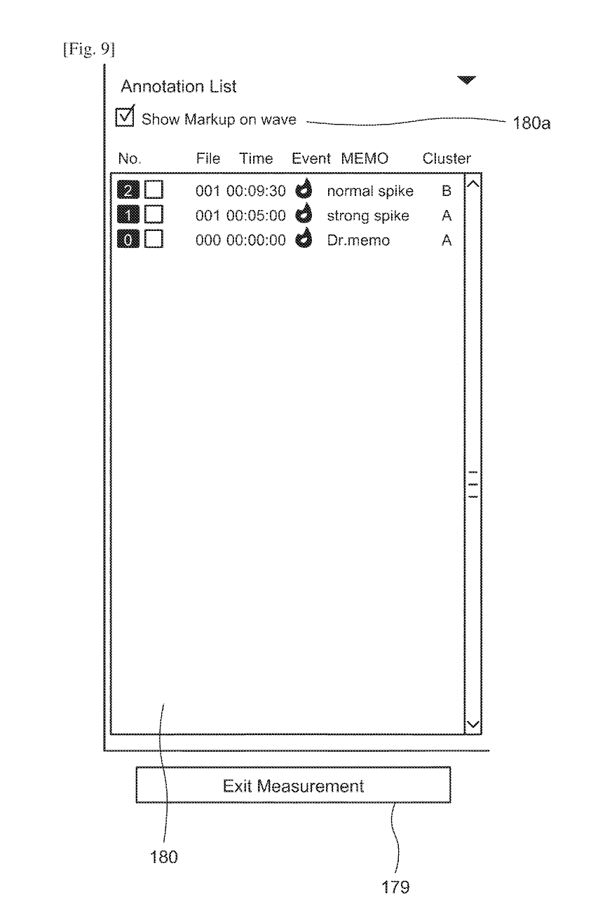

FIG. 8 is a diagram illustrating a display state of the area 201A at the left side of the screen 201 immediately after “spike” is selected in the pop-up window 115 and text, “normal spike”, is input. When an “OK” button is selected in the pop-up window 115 illustrated as an example in FIG. 6, the pop-up window 115 is closed, and the annotation 110a-2 is displayed at a corresponding time position in the display area 110 as illustrated in FIG. 8. In this example, in association with the annotation number, “2”, the attribute icon representing “spike”, and the text information, “normal spike”, are displayed. Further, simultaneously therewith, the value in the counter box 118 is incremented.

Further, an attribute icon 106-2 is displayed near the mark 103a-2 for emphasized display. In this example, an attribute icon 106-1 is displayed near the mark 103a-1, but as described above, display or non-display of the attribute icons 106-1 and 106-2 is selectable. At the marks 103a-1 and 103a-2, as lines intersecting the time axis 112, lines 117-1 and 117-2 orthogonal to the time axis 112 at corresponding times on the time axis 112 are displayed. These lines 117-1 and 117-2 are an example of the emphasized display.

FIG. 9 is a diagram illustrating the annotation list 180. By the addition of the annotation corresponding to the mark 103a-2 in the area 201A illustrated in FIG. 8, the annotation list 180 is updated. The memo, “normal spike”, is added to the annotation number, “2”.

Similarly, hereinafter, every time a spot to be noted on signal waveforms is specified in the area 201A during measurement, the specified spot is displayed with emphasis, and an annotation is displayed along the time axis 112 in the display area 110. In the area 201B, information on the annotation is added to the annotation list 180 in order.

The display of the annotation numbers in the annotation list 180 and in the area 201A for display of signal waveforms is not essential, and the annotation numbers may be not used. Any information enabling the assigned annotations to be identified may be used as identification information, instead of the annotation numbers. For example, an attribute icon and an attribute character string (such as “strong spike”) may be displayed near the time axis 112 in association with a time. Further, a file number may be displayed together therewith in the area 201A.

When the measurement is ended by the selection (depression) of the “Exit Measurement” button 179, the spots specified in the display areas 101 to 103 are stored in the specified destination, in association with the signal waveforms (biological data). The annotations displayed in the display area 110 at the time positions corresponding to the specified spots are also stored in association with the annotation numbers and the times. Related information, such as the counter value in the counter box 118, and contents of the annotation list 180, is also stored. By the storage of these pieces of display information, even if the measurer and the analyst are different individuals, the analyst is able to easily recognize the spots to be noted and perform analysis.

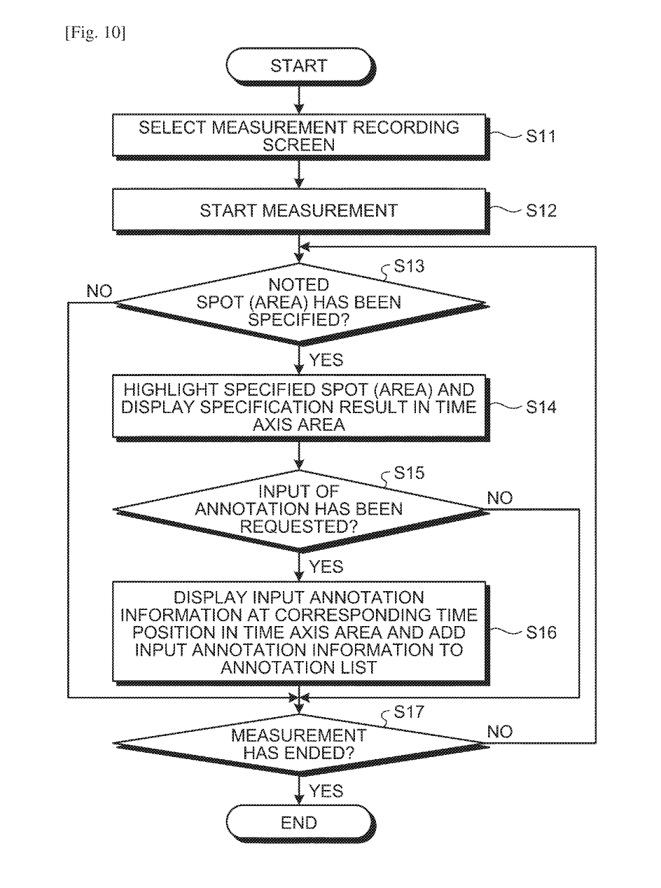

FIG. 10 is a flow chart illustrating an example of information display processing executed by the information processing apparatus 50 at a measurement recording stage. When the “Measurement Recording” button 204-1 is selected on the start screen 204 illustrated in FIG. 4 (Step S11), measurement is started, and waveforms of plural signals are displayed in synchronization with one another along the same time axis in the area 201A of the measurement recording screen 201 (see FIG. 5) (Step S12).

The information processing apparatus 50 determines whether or not a spot to be noted (also referred to as “noted spot”) has been specified on the signal waveforms being displayed (Step S13). If a noted spot has been specified (YES at Step S13), the specified spot is displayed with emphasis in the display area for the signal waveforms (display areas 101 to 103), and a result of the specification is displayed at a corresponding time position in a time axis area (display area 110) (Step S14). The result of the specification includes information indicating that the specification has been made, or identification information of the specification. Simultaneously with the display of the result of the specification in the time axis area, or at a time around the time of that display, whether or not input of an annotation has been requested is determined (Step S15). If input of an annotation has been requested (YES at Step S15), the input annotation is displayed at the corresponding time position in the time axis area, and information corresponding to that annotation is added to the annotation list (Step S16). Thereafter, whether or not a measurement ending command has been input (the “Exit Measurement” button 179 has been pressed down) is determined (Step S17). If a noted spot has not been specified (NO at Step S13), or if input of an annotation has not been requested (NO at Step S15), the processing is skipped to Step S17 and whether the measurement has ended is determined. If it is determined that the measurement has not ended (NO at Step S17), Step S13 to Step S16 are repeated. If it is determined that the measurement has ended (YES at Step S17), the measurement processing is ended.

By this information display method, a measurement recording screen that is high in visibility of signal information is provided when signals from plural sensors are collected.

### Operation in Analysis

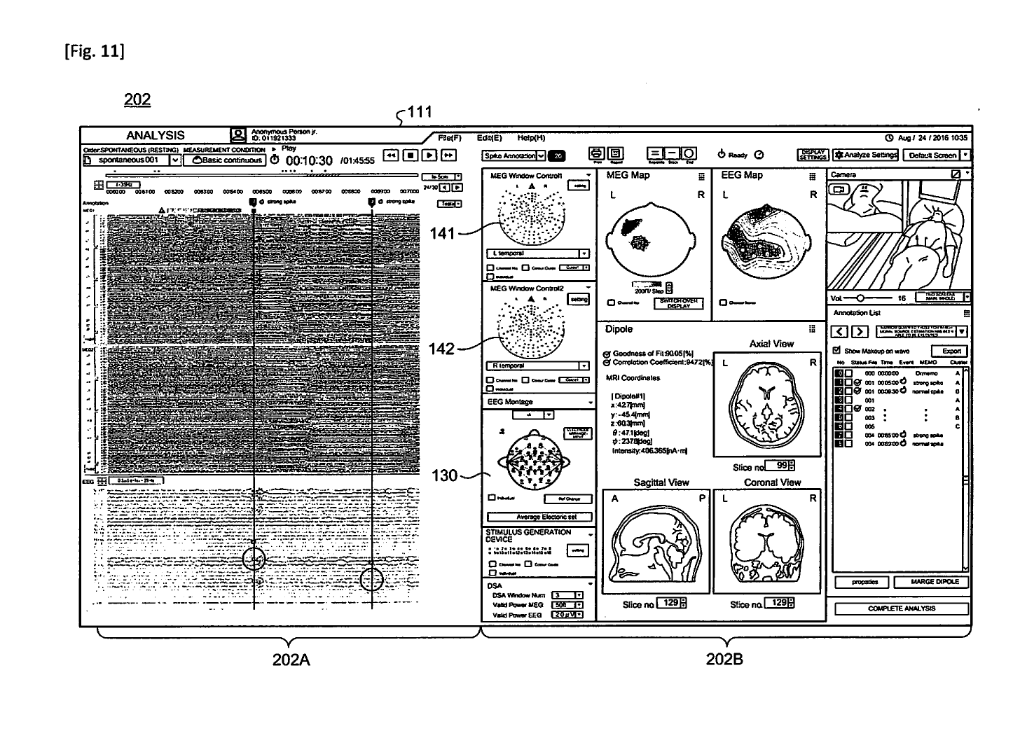

FIG. 11 illustrates an example of the screen of the information processing apparatus 50 at the time of analysis. An analysis screen (or also simply referred to as “screen”) 202 is displayed by an “Analysis” button 204-2 being selected on the start screen 204 in FIG. 4. The tab 111 on the screen 202 displays therein that the screen 202 is an “Analysis” screen.

The information processing apparatus 50 according to this embodiment has a function of executing control to display this analysis screen 202 on the display device 28 (see FIG. 1). In the example of FIG. 11, the analysis screen 202 has an area 202A where waveforms (corresponding to biological data) representing temporal change of three sets of biological signals that have been recorded are displayed together with annotations, and an area 202B where analysis information is displayed. The area 202A where the waveforms of the recorded biological signals and the annotations are displayed is arranged at a left side of the screen 202 as viewed from the measurer, and the area 202B for analysis display is arranged at a right side of the screen 202 as viewed from the measurer. This arrangement is for improved operability with the mouse or the like and improved efficiency of eye movements when results of analysis are checked or confirmed in the area 202B while noted spots on the signal waveforms are selected in the area 202A, at the time of analysis.

In this example, the signal waveforms and the annotations are displayed in the area 202A in a layout similar to that in the area 201A (see FIG. 8) of the measurement recording screen 201. Specifically, waveforms of electroencephalography signals and annotations are displayed in the display area 103, and waveforms of magnetoencephalography signals are displayed in each of the display areas 101 and 102. In the area 202B, controllers, images, and the like, for analysis of the plural signal waveforms displayed in the area 202A are displayed.

At corresponding positions (on the immediate right) of the display area 101, the display area 102, and the display area 103, a magnetoencephalographic distribution diagram 141, a magnetoencephalographic distribution diagram 142, and an electroencephalographic distribution diagram 130 that function as controllers illustrated in the area 201B (see FIG. 7) of the measurement recording screen 201 are provided. By selecting sensors represented by dots on the magnetoencephalographic distribution diagram 141, an analyst is able to limit the magnetoencephalography signals to be displayed in the display area 101. Further, by selection of sensors represented by dots on the magnetoencephalographic distribution diagram 142, the magnetoencephalography signals to be displayed in the display area 102 are able to be limited. Furthermore, by selection of electrodes on the electroencephalographic distribution diagram 130, the electroencephalography signals to be displayed in the display area 103 are able to be limited. Therefore, eye movements of the analyst become efficient, and as a result, efficiency of the analytic work is able to be improved.

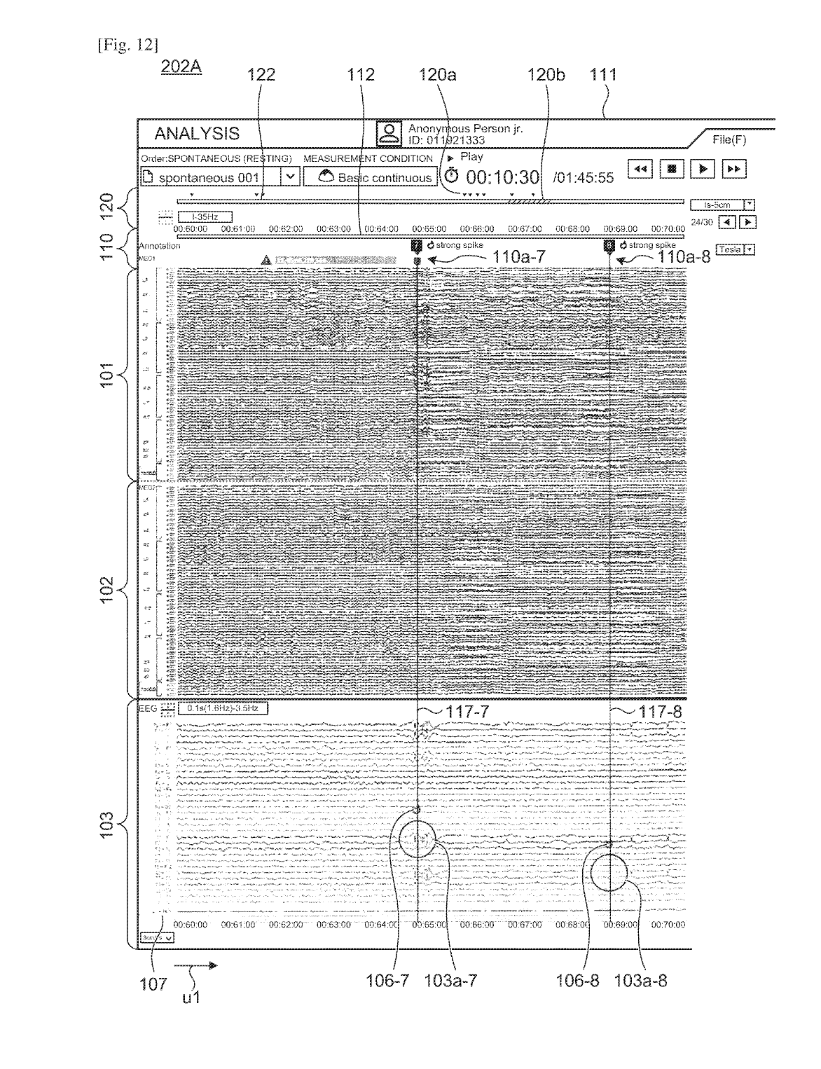

FIG. 12 is an enlarged view of the area 202A at the left side illustrated in FIG. 11. The area 202A has the display area 110 and the display area 120, where time information for measurement is displayed, and the display areas 101 to 103 where recorded signal waveforms (biological data) are parallelly displayed.

In the display area 110, the time axis 112 indicating elapse of time in recording, and annotations assigned along the time axis 112 are displayed. A state where an annotation 110a-7 of an annotation number, “7”, and an annotation 110a-8 of an annotation number, “8”, have been displayed is illustrated in FIG. 12. In the display area 120, a time axis 122 representing the whole recording time period is displayed. Along the time axis 122, pointer marks 120a indicating time positions assigned with annotations are displayed side by side. Further, on the time axis 122, a time zone 120b representing a time zone within the whole recording time period is displayed, the time zone being for the signal waveforms being displayed in the display areas 101 to 103. The time zone 120b is represented by a hatched area in FIG. 12. By this display, the analyst is able to intuitively understand at which stage in the measurement recording the signal waveforms being displayed (being analyzed) were acquired.

After opening the analysis screen 202, the analyst is able to cause signal waveforms of a desired time zone to be displayed in the display areas 101 to 103 by, for example, dragging the time zone 120b on the time axis 122. Or, by selection of a desired annotation from the annotation list 180 in the area 202B described later, signal waveforms around and including that annotation are able to be displayed in the display areas 101 to 103.

In the display areas 101 to 103 illustrated in FIG. 12, signal waveforms that have been recorded are parallelly displayed by being arranged horizontally. Further, in the display areas 101 to 103, spots that have been specified during the recording are displayed with emphasis. Specifically, two specified spots are displayed with emphasis with a mark 103a-7 and a mark 103a-8, and an attribute icon 106-7 and an attribute icon 106-8 provided near these marks.

Further, vertical lines 117-7 and 117-8 are displayed, which are provided to extend in a direction intersecting (a direction orthogonal to, in this example) a measurement time direction (the direction u1) and which join the mark 103a-7 and mark 103a-8 with the annotation 110a-7 and annotation 110a-8 indicating time positions corresponding to these marks, respectively. By lines 117 (corresponding to the line 117-7 and line 117-8 in the example illustrated in FIG. 12) being displayed, for example, the annotations on the time axis 112 related to the specified spots in the display area 103 and the signal waveforms in the display areas 102 and 101, which are signal display areas different from the display area 103, are able to be visually recognized easily. Further, by the line 117-7 or line 117-8 being selected, the signal waveforms corresponding to a certain time period around the selected time are displayed enlarged. This processing will be described later.

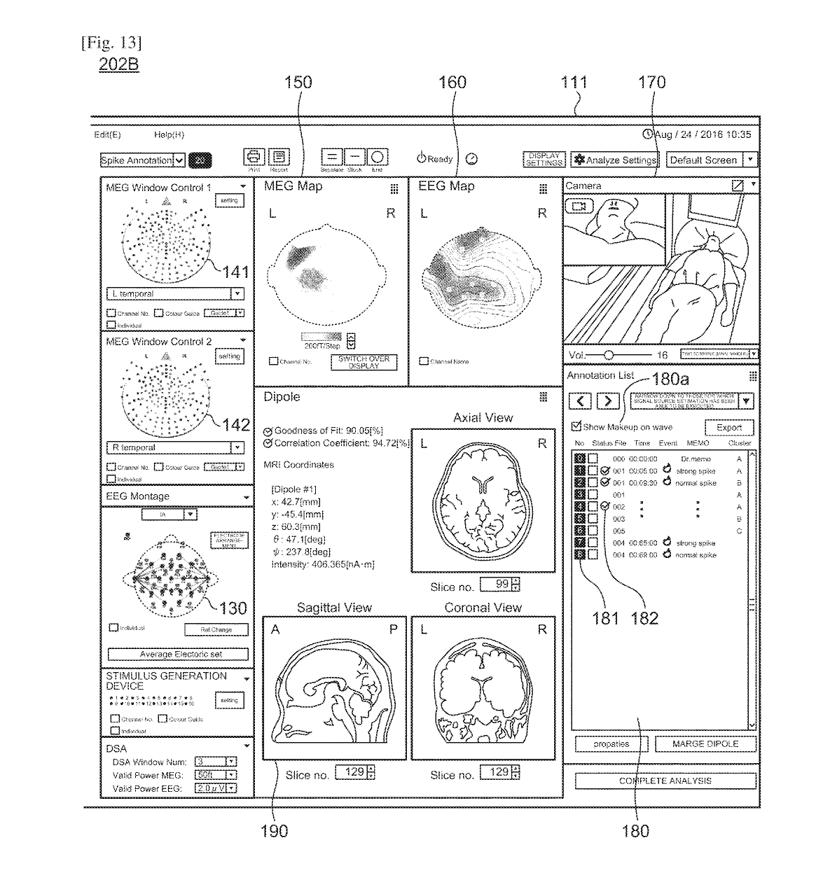

FIG. 13 is an enlarged view of the area 202B at the right side, the area 202B representing the same time zone as that in FIG. 12. In the area 202B at the right side, the magnetoencephalographic distribution diagram 141 corresponding to the signal waveforms being displayed in the display area 101 (see FIG. 12), the magnetoencephalographic distribution diagram 142 corresponding to the signal waveforms being displayed in the display area 102 (see FIG. 12), and the electroencephalographic distribution diagram 130 corresponding to the signal waveforms being displayed in the display area 103 (see FIG. 12) are displayed. Further, an isomagnetic field diagram 150 of a magnetoencephalograph (MEG), a map area 160 of an electroencephalograph (EEG), and a display window 190 for tomographic images of the brain of the person to be measured, the tomographic images having been acquired separately by examination through magnetic resonance imaging (MRI), are displayed. In the isomagnetic field diagram 150, source regions and sink regions of the magnetic field are displayed in different colors, and flow directions of electric currents are visually understood therefrom. The isomagnetic field diagram 150 and the map area 160 are information acquired after the measurement is completed, and the tomographic images of MRI are information acquired separately by examination.

In the monitoring window 170, video of the person to be measured at the time of measurement is displayed in synchronization with the times, at which the signal waveforms in the display areas 101 to 103 were acquired. The analyst is able to analyze the signal waveforms while checking the state of the person to be measured, by looking at the monitoring window 170.

All of annotations assigned in measurement recording are listed in the annotation list 180. The annotation list 180 includes information on the annotations (attribute icons, text input information, and the like), the information having been added in association with annotation numbers 181. In the annotation list 180 on the analysis screen 202, the information on the added annotations is displayed in, for example, ascending order (with older data being displayed up). This mode of display is just an example, and the display is not limited to this mode. For example, similarly to the measurement recording screen 201, the use of annotation numbers is not essential, and annotations may be identified by combinations of times, file names, attributes, and the like. Further, the order of display of the information on the annotations included in the annotation list 180 may be changed, and the information may be sorted by item. By a desired one of the annotation numbers 181 or the lines being clicked, signal waveforms of a predetermined time zone including the time position assigned with that annotation are able to be displayed in the display areas 101 to 103 of FIG. 12.

In contrast to the annotation list on the measurement recording screen 201 (see FIG. 5, FIG. 7, and FIG. 9), when the analyst checks the signal waveforms of each annotation included in the annotation list by display of the signal waveforms, and signal source estimation processing is finally executed thereon, an estimation completion mark 182 is displayed in the annotation list 180 for that annotation subjected to the estimation.

In a selection box 180a, display/non-display of the annotations is selected. When non-display is specified in the selection box 180a, the attribute icons 106-7 and 106-8 in the display area 103 in FIG. 12 disappear. This may be modified such that non-display of the marks 103a-7 and 103a-8 for emphasized display is made selectable in the selection box 180a.

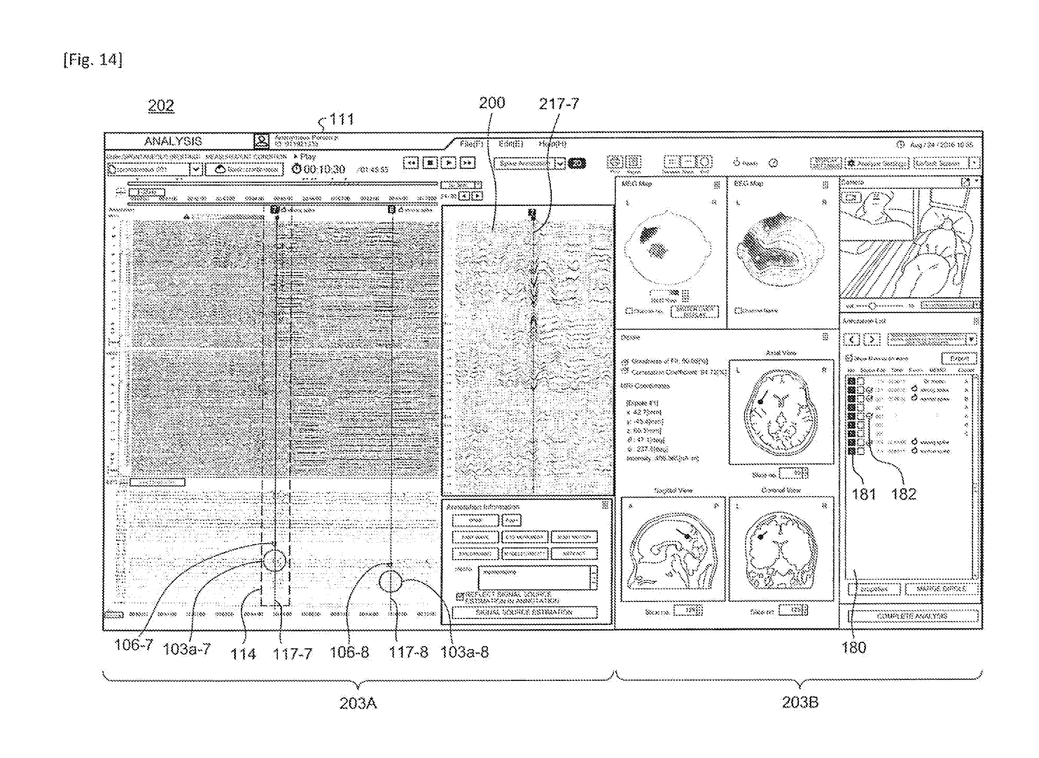

FIG. 14 is a diagram illustrating a display state immediately after the line 117-7 is selected on the analysis screen 202 in FIG. 12. When the analyst selects the line 117-7 by, for example, double-clicking, for analyzing the waveforms in the area of the mark 103a-7, signal waveforms in the area near and including the mark 103a-7 are displayed enlarged in an enlarged display area 200. In the example illustrated in FIG. 14, signal waveforms displayed in a certain time range of the width of an area 114, that is, signal waveforms from sensors in the area around and including the marked spot, are displayed enlarged, together with a line 217-7 indicating the time position.

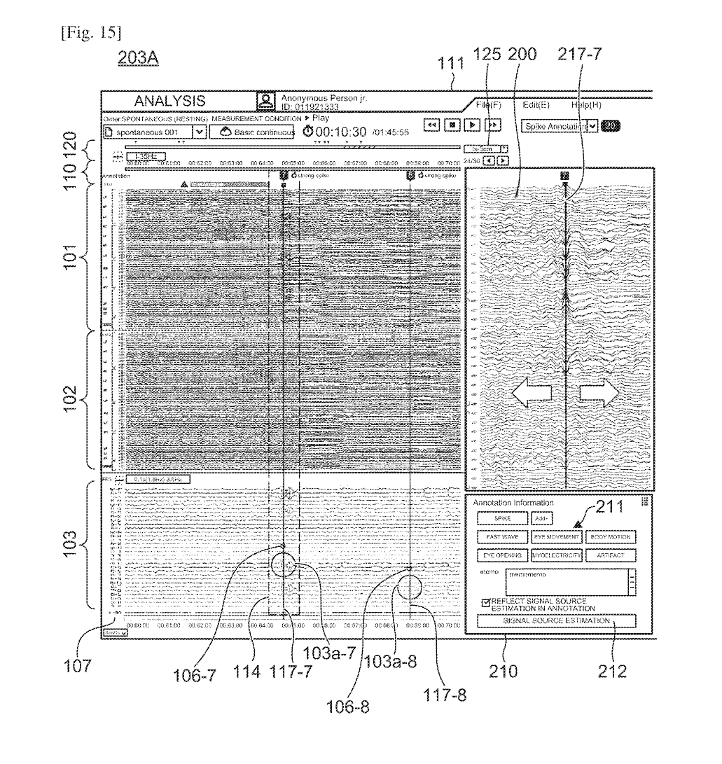

FIG. 15 is an enlarged view of an area 203A (a display area for signal waveforms) illustrated at a left side in FIG. 14. By displaying the signal waveforms enlarged in the enlarged display area 200, the analyst is able to recheck the validity of the spot marked during the recording, or check any waveform portion that was not checked during the measurement recording. For example, by the line 217-7 being dragged to the left or right, a disturbance in the waveforms, or an accurate location of a singular point is able to be identified, and a change may be made. The mark 103a-7 and the attribute icon 106-7 for emphasized display in the display area 103 may be reflected in the enlarged display area 200. However, since they may obstruct visual recognition upon precise determination of a singular point of amplitude, if the mark 103a-7 and the attribute icon 106-7 are displayed in the enlarged display area 200, display and non-display thereof is desirably made selectable.

The type of signal waveforms (electroencephalographic waveforms or magnetoencephalographic waveforms) to be displayed in the enlarged display area 200, or a channel range may also be specified. For example, the analyst may move her line of sight upward on the screen 202 to check whether the waveforms in the display area 101 or 102 for magnetoencephalographic waveforms have singular points of amplitude. In this case, by input, in a box 125, of information specifying a targeted channel area in the display area 101 or 102, the magnetoencephalographic waveforms of that channel area corresponding to the line 217-7 are able to be displayed enlarged in the enlarged display area 200.

A confirmation window 210 is displayed below the enlarged display area 200. The confirmation window 210 includes attribute buttons 211 for the signal waveforms and a signal source estimation button 212. The attribute buttons 211 correspond to attribute information included in the pop-up window 115 on the measurement recording screen 201, and when any attribute assigned at the time of recording is incorrect, a correct attribute is able to be selected by selection from the attribute buttons 211. When the correct position on the signal waveforms and the selection of the attribute have been confirmed, by the estimation button 212 being clicked, signal source estimation is executed. As described later, an estimated signal source is able to be displayed superimposed on a tomographic image corresponding to the estimated signal source, the tomographic image being among plural tomographic images (biological tomographic images) of the brain of the person to be measured, which have been acquired by magnetic resonance imaging (MRI). The signal source estimation may be executed by the information processing apparatus 50 according to this embodiment, or may be executed by an external device.

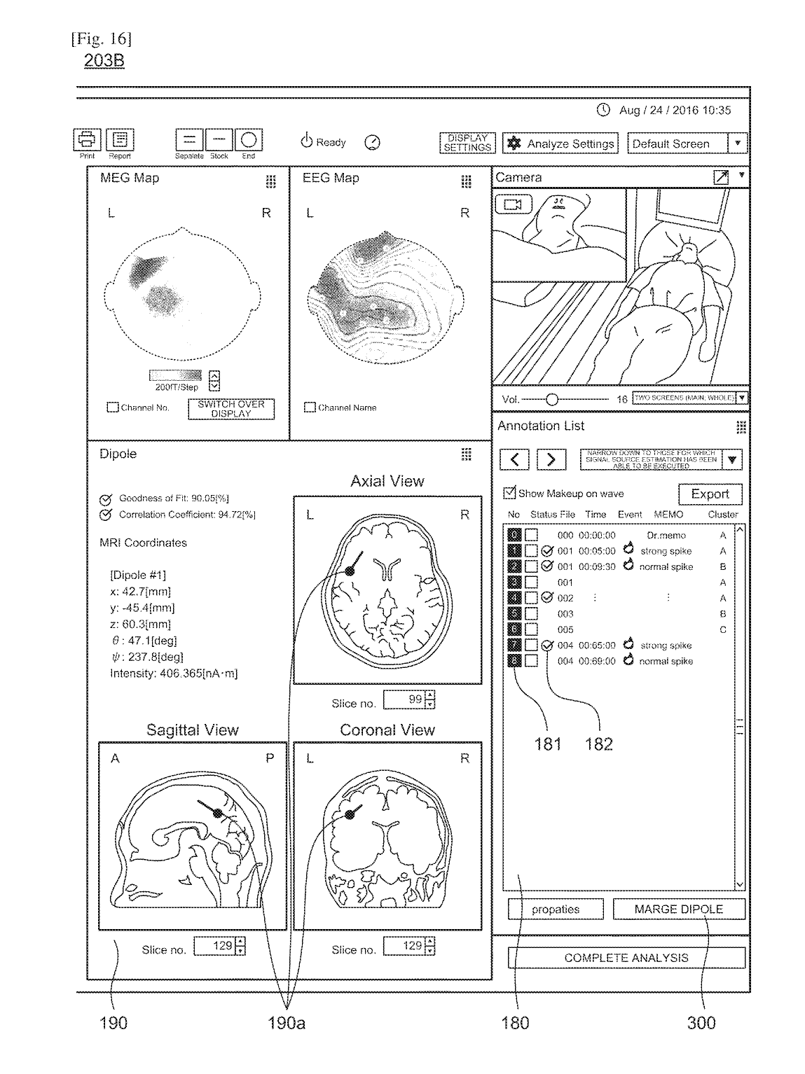

FIG. 16 is an enlarged view of an area 203B illustrated at a right side in FIG. 14. When the position and the attribute of the mark are confirmed and the signal source estimation button 212 is selected in FIG. 15, a signal source is estimated, and the estimation completion mark 182 is assigned to the corresponding annotation number 181 (in this example, the annotation number, “7”) in the annotation list 180 of FIG. 16. Further, on the MRI tomographic images in the display window 190, an estimation result 190a for a dipole is displayed.

There are two methods of updating the annotation list 180 when the position of a mark in the display areas 101 to 103 or the content of an annotation is changed by the analyst. They are: a method of reflecting only the latest update information by the analyst in the annotation list 180; and a method of adding the latest update information as new annotation information while maintaining the annotation information added at the time of measurement recording. When the latter method is adopted, for example, a branch number from the annotation number assigned at the time of recording may be assigned as annotation identification information. In this case, the new annotation information may be added also in the display area 110, and the added annotation may be displayed in a different color along the time axis.

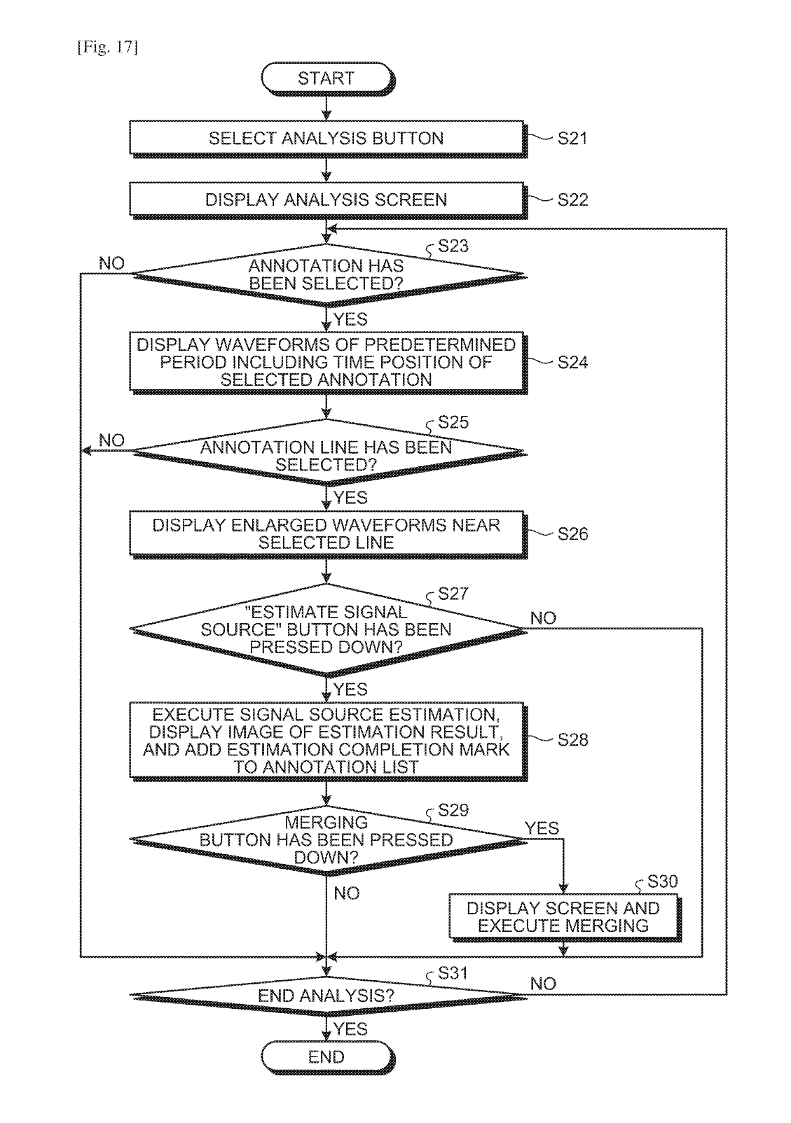

FIG. 17 is a flow chart illustrating an example of information display processing executed by the information processing apparatus 50 at an analysis stage. The following information display processing is executed by: the control unit 250 of the information processing apparatus 50 determining input operation. When the “Analysis” button 204-2 is selected on the start screen 204 (see FIG. 4) (Step S21), analysis is started, and the analysis screen 202 (see FIG. 11) is displayed (Step S22). The initial analysis screen 202 may be a blank screen having no signal waveforms displayed thereon, or may have signal waveforms of a certain time range at the head or end of the recording. When the analysis screen 202 is displayed, whether or not a specific annotation has been selected is determined (Step S23). The selection of an annotation may be selection of a specific annotation number or line in the annotation list 180 (see FIG. 13), or specification of a time position by operation on the time zone 120b on the time axis 122 in the display area 120 (see FIG. 12). When an annotation has been selected (YES at Step S23), signal waveforms corresponding to a predetermined time period including the time position of the selected annotation are displayed (Step S24).

Thereafter, whether or not the line 117 indicating a time position of a mark for emphasized display (corresponding to the line 117-7 or the line 117-8 in the example illustrated in FIG. 12) has been selected is determined (Step S25). When the line 117 has been selected (YES at Step S25), signal waveforms of a certain time range including the selected line are displayed enlarged in the enlarged display area 200 (see FIG. 14) (Step S26). The signal waveforms that are displayed enlarged may be signal waveforms of all of the channels. Further, signal waveforms acquired in a certain range of channels including the channel/channels where the marked signal waveform/waveforms has/have been acquired may be displayed enlarged. Furthermore, by input of a targeted channel area in the box 125 (see FIG. 15), the enlarged display of the signal waveforms in the mark for emphasized display and signal waveforms of the same type near that mark may be changed to enlarged display of signal waveforms of a different type or different types at the same time position as the mark for emphasized display. For example, when a mark for emphasized display has been assigned to electroencephalography signal waveforms, magnetoencephalography signal waveforms at the same time position may be displayed enlarged. In this case, presence or absence of input of specification of: the type/types of signal waveforms or channel range/ranges, which is/are desired to be displayed enlarged, is determined.

Next, whether or not the signal source estimation button 212 (see FIG. 15) has been pressed down is determined (Step S27). When the signal source estimation button 212 has been pressed down (YES at Step S27), calculation for signal source estimation is executed, a result of the estimation is displayed on the MRI tomographic images, and the estimation completion mark 182 is added in the annotation list 180 (see FIG. 16) (Step S28). If the information processing apparatus 50 receives depression of a merging button 300 arranged below the annotation list 180 (YES at Step S29), the information processing apparatus 50 executes merging processing of making a target spot being a cause of a case easy to be identified by displaying a list of specified spots, for which estimation has been completed (Step S30). This merging processing will be described in detail later. If depression of the merging button 300 is not received (NO at Step S29), or after Step S30; whether or not an analysis ending command has been input is determined (Step S31). If an annotation has not been selected (NO at Step S23), if the line 117 for enlarged display has not been selected (NO at Step S25), or if the signal source estimation button 212 has not been pressed down (NO at Step S27), the processing is skipped to Step S31, and whether the analysis is to be ended is determined. Until the analysis ending command is input (YES at Step S31), Step S23 to Step S30 are repeated.

Between Step S26 and Step S27, whether or not an annotation has been changed may be determined. If an annotation has been changed, the change is reflected in the annotation list 180, and the processing is advanced to the determination in Step S27.

By the above described display processing operation, information display excellent in visibility and operability is implemented.

### Merging Processing

In the merging processing, the display control unit 251 (see FIG. 3) displays a screen having a first display area and a second display area. In this display processing, the display control unit 251 executes control to parallelly display plural biological signal groups in the first display area and displaying biological tomographic images, on which the signals sources have been displayed superimposed, in the second display area, the plural biological signal groups having one-to-one correspondence to plural annotations, for which signal source estimation has been completed, each of the plural biological signal groups representing a waveform or waveforms of one or more biological signals at the position or range indicated by the annotation. A “biological signal group” corresponds to “partial data”, and represents biological data corresponding to a partial time period. In this example, the display control unit 251 displays the plural biological signal groups side by side from a group whose measurement is performed earlier, to a group whose measurement is performed later. Further, if any one biological signal group is selected from the plural biological signal groups, the display control unit 251 displays the biological tomographic images including the signal source corresponding to the selected biological signal group, and displays information indicating the signal source with the information being superimposed on the biological tomographic images. Furthermore, if a signal source corresponding to a biological signal group that has not been selected is present on the biological tomographic images including the signal source corresponding to the selected biological signal group, the display control unit 251 displays information indicating that signal source corresponding to the non-selected biological signal group with the information also being superimposed on the biological tomographic images. Hereinafter, specific content of the merging processing will be described.

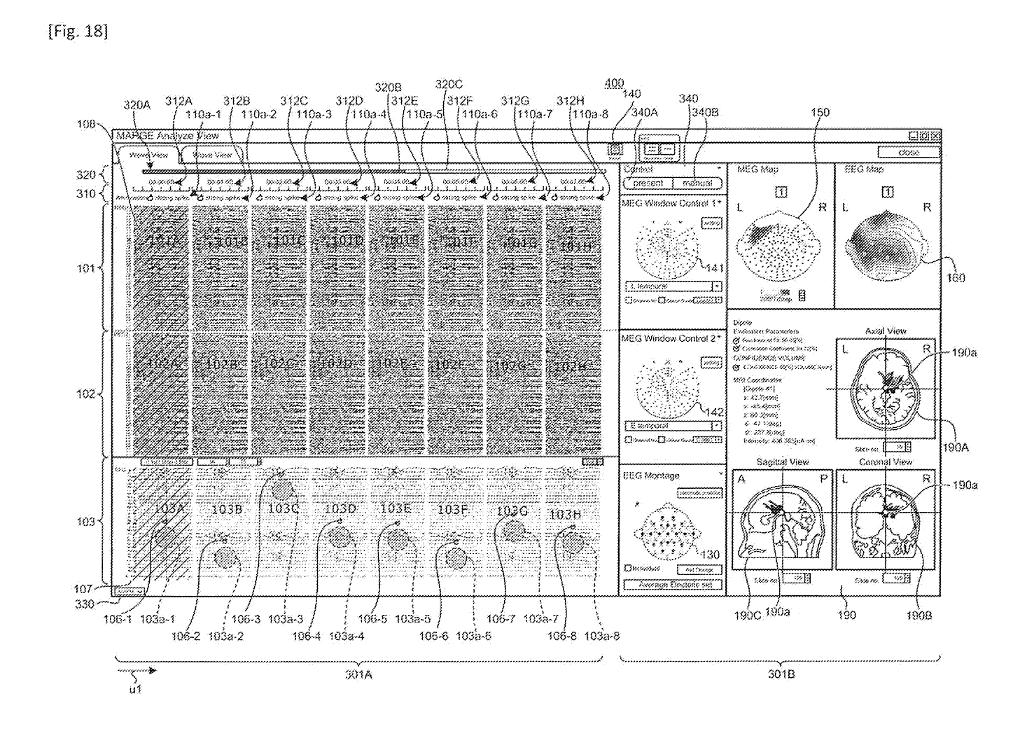

In this embodiment, if the display control unit 251 receives depression of the merging button 300 arranged below the annotation list 180 illustrated in FIG. 16, the display control unit 251 executes control to display a screen 400 as illustrated in FIG. 18 on the display device 28. The screen 400 includes an area 301A, which is an example of the first display area, and an area 301B, which is an example of the second display area. In the area 301A, for each annotation that has been assigned with the estimation completion mark 182, among the plural annotations displayed in the annotation list 180 illustrated in FIG. 16, a biological signal group in a range corresponding to that annotation (in a range divided by a time period), among biological data that have been recorded, is displayed. In this example, a biological signal group including two sets of magnetoencephalography signals and one set of electroencephalography signals is illustrated as an example. Since the biological signal group is displayed in a strip form as illustrated in FIG. 18, hereinafter, a biological signal group in a range divided by a time period may be referred to as “strip waveform”. In the other area 301B, analysis information on the strip waveforms being displayed in the area 301A is displayed. By the area 301A, in which the respective strip waveforms are displayed, being arranged at the left side, and the area 301B, in which the analysis information is displayed, being arranged at the right side, the same layout as the analysis screen 202 illustrated in FIG. 11 and FIG. 14 is obtained, and workability for the analyst is improved. If the display control unit 251 receives depression of a predetermined button (a “Return” button, for example), the display control unit 251 executes control to return to display of the analysis screen 202.

In the area 301A at the left side on the screen 400 illustrated in FIG. 18, plural strip waveforms having one-to-one correspondence to the plural annotations assigned with the estimation completion marks 182 in FIG. 16 are displayed parallelly along an overall time axis 320A. In this example, plural strip waveforms having one-to-one correspondence to plural annotations of the same attribute (“spike” in the example of FIG. 18) are parallelly displayed. When, for example, the analyst presses down the merging button 300, the analyst is able to perform operation of specifying any one of the attributes. When the display control unit 251 receives depression of the merging button 300 and the operation of specifying an attribute, the display control unit 251 is able to execute control to parallelly display plural strip waveforms having one-to-one correspondence to plural annotations representing the specified attribute, among the plural annotations that have been assigned with the estimation completion marks 182 in FIG. 16. However, not being limited to this example, when the display control unit 251 receives depression of the merging button 300, the display control unit 251 may also execute control to parallelly display plural strip waveforms having one-to-one correspondence to the plural annotations that have been assigned with the estimation completion marks 182 in FIG. 16, regardless of whether the attributes are the same.

On a left side of the display areas 101, 102, and 103 illustrated in FIG. 18, a channel number 107 of each of the waveforms is displayed, and in this example, waveforms from all of the sensors, or arbitrary sensors selected (specified) by the analyst are displayed. In the display areas 101, 102, and 103, strip waveforms, each of which is over two-seconds around a marked spot among biological data that have been recorded, are parallelly displayed.

In the example of FIG. 18, eight strip waveforms are parallelly displayed. A hatched area 108 in FIG. 18 represents one strip waveform in this example. The number of strip waveforms may be arbitrarily modified, and may be less than eight or greater than eight. On the screen 400, for explanation of the types of biological signals included in the strip waveforms in this example, reference signs have been assigned to the corresponding display areas. The top left display area on the screen 400 is a display area 101A, and the display areas in order from the top to the bottom of the screen 400 are the display area 101A, a display area 102A, and a display area 103A. Further, rightward on the screen 400, the reference signs are assigned one by one to the display areas by change of the symbol, “A”, to “B, C, D, . . . ”. Hereinafter, by use of these reference signs, formation of the strip waveforms in this example will be described. The first strip waveform from the left on the screen 400 (the strip waveform added with the hatched area 108 in FIG. 18) is formed of a set of: waveforms displayed in the display area 101A; waveforms displayed in the display area 102A; and waveforms displayed in the display area 103A. Similarly, the second strip waveform is formed of a set of: waveforms displayed in a display area 101B; waveforms displayed in a display area 102B; and waveforms displayed in a display area 103B; the third strip waveform is formed of a set of: waveforms displayed in a display area 101C; waveforms displayed in a display area 102C; and waveforms displayed in a display area 103C; the fourth strip waveform is formed of a set of: waveforms displayed in a display area 101D; waveforms displayed in a display area 102D; and waveforms displayed in a display area 103D; the fifth strip waveform is formed of a set of: waveforms displayed in a display area 101E; waveforms displayed in a display area 102E; and waveforms displayed in a display area 103E; the sixth strip waveform is formed of a set of: waveforms displayed in a display area 101F; waveforms displayed in a display area 102F; and waveforms displayed in a display area 103F; the seventh strip waveform is formed of a set of: waveforms displayed in a display area 101G; waveforms displayed in a display area 102G; and waveforms displayed in a display area 103G; and the eighth strip waveform is formed of a set of: waveforms displayed in a display area 101H; waveforms displayed in a display area 102H; and waveforms displayed in a display area 103H. In this example, sizes of all of the strip waveforms in a width direction thereof are the same. By parallel display of, for example, disturbances in the waveforms or singular points of amplitude (which will, hereinafter, be referred to as “singular points”), the respective strip waveforms are able to be compared with one another.

The respective strip waveforms are arranged, from the left to the right on the screen 400, from the one whose measurement is performed earlier, to the one whose measurement is performed later, and thus trends of waveforms (for example, spike waveforms) of singular points according to the measurement time are able to be compared with one another. Further, on the screen 400 illustrated in FIG. 18, a display area 310 arranged above the display area 101 has time axes 312A to 312H displaying thereon the times of measurement of the strip waveforms. By check of the times being displayed on the respective time axes, frequency of spikes (time interval) is able to be visually recognized. Annotations 110a-1 to 110a-8 are displayed at corresponding time positions on the respective time axes.

Further, in the display areas 101 to 103, spots that have been specified are displayed with emphasis with marks and attribute icons near the marks. In the example of FIG. 18: in the display area 103A of the first strip waveform, a mark 103a-1 and an attribute icon 106-1 are displayed; in the display area 103B of the second strip waveform, a mark 103a-2 and an attribute icon 106-2 are displayed; in the display area 103C of the third strip waveform, a mark 103a-3 and an attribute icon 106-3 are displayed; in the display area 103D of the fourth strip waveform, a mark 103a-4 and an attribute icon 106-4 are displayed; in the display area 103E of the fifth strip waveform, a mark 103a-5 and an attribute icon 106-5 are displayed; in the display area 103F of the sixth strip waveform, a mark 103a-6 and an attribute icon 106-6 are displayed; in the display area 103G of the seventh strip waveform, a mark 103a-7 and an attribute icon 106-7 are displayed; and in the display area 103H of the eighth strip waveform, a mark 103a-8 and an attribute icon 106-8 are displayed; and thereby the specified spots are displayed with emphasis in the strip waveforms thereof.

Further, in FIG. 18, in the display area 320 arranged above the display area 310, a displayed page position of the whole strip waveforms being displayed is displayed. For example, if the number of annotations assigned with the estimation completion marks 182, among the plural annotations displayed in the annotation list 180 illustrated in FIG. 16, exceeds the number displayable per screen (per page), that collection of strip waveforms extends over plural pages. In this case, in the display area 320, information indicating the position of the page currently being displayed, in relation to the whole collection including the plural pages, is displayed. In this example, the overall time axis 320A is arranged, and a display area 320B illustrated in gray in FIG. 18 indicates the position of the strip waveforms being displayed on the screen (the position in relation to the whole time period), and an area 320C illustrated in blank indicates the position of strip waveforms that are not being displayed on the screen 400. When the display control unit 251 receives an operation for change of the position of the gray display area 320B, the display control unit 251 executes control to switch over the display to that of a strip waveform group corresponding to the changed display area 320B.

Further, a display area 330 arranged at the lower left of the screen 400 in FIG. 18 is a display area for reception of input for change of a display width per unit time (for example, one second) for the strip waveforms. According to the input received through the display area 330, the display control unit 251 is able to change the display width per unit time for the strip waveforms. For example, when the position of a spike is desired to be enlarged, or when an area around the spike is desired to be overlooked; by change of the display width, display specialized for information more desired to be noted is enabled.

In the area at the right side of the screen 400, magnetoencephalographic distribution diagrams 141 and 142 corresponding to the signal waveforms being displayed in the display areas 101 and 102, and an electroencephalographic distribution diagram 130 corresponding to the signal waveforms being displayed in the display area 103 are displayed. Further, an isomagnetic field diagram 150 of a magnetoencephalograph (MEG), a map area 160 of an electroencephalograph (EEG), and a display window 190 for tomographic images of the brain of the person to be measured, the tomographic images having been acquired by magnetic resonance imaging (MRI), are displayed.

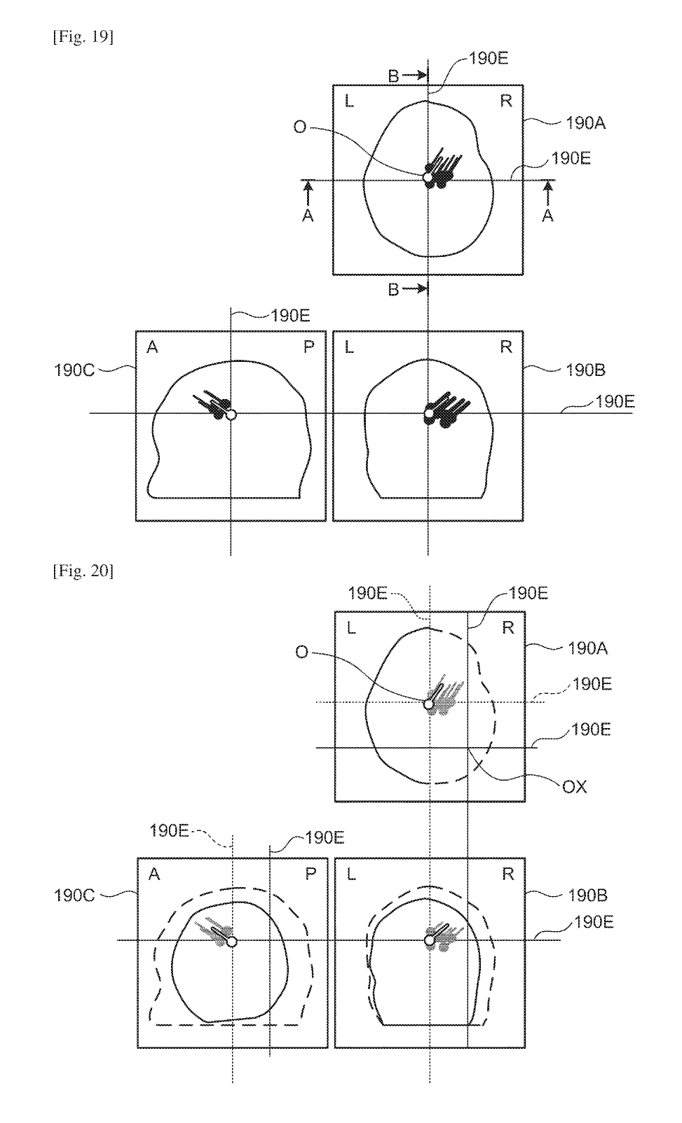

The tomographic images in the display window 190 will now be described. As illustrated in FIG. 19, the tomographic images in the display window 190 are formed of: a slice image 190A from the top; a slice image 190B from the back; and a slice image 190C from the side. The slice images 190A to 190C are positionally linked with one another in three-dimensional directions. Reference lines 190E are displayed to extend over the respective slice images, and intersection points O of the respective reference lines 190E indicate sliced positions of the respective slice images. In this example, the slice image 190B is an image of a cross section viewed in an A-direction illustrated in FIG. 19, the cross section being at the position of the reference line 190E that is in a horizontal direction (left-right direction) illustrated in the slice image 190A (the position also corresponding to the position of the reference line 190E that is in an up-down direction illustrated in the slice image 190C). The slice image 190C is an image of a cross section viewed in a B-direction illustrated in FIG. 19, the cross section being at the position of the reference line 190E that is in an up-down direction illustrated in the slice image 190A (the position also corresponding to the position of the reference line 190E that is in an up-down direction illustrated in the slice image 190B). The slice image 190A is an image of a cross section viewed from an upper side of the slice image 190B, the cross section being at the position of the reference line 190E that is in a horizontal direction illustrated in the slice image 190B (the position also corresponding to the position of the reference line 190E that is in a horizontal direction illustrated in the slice image 190C).

As illustrated in FIG. 20, where the reference lines 190E at the currently displayed positions are represented by solid lines, when the analyst moves the intersection point O to OX by dragging it with a mouse, the reference lines 190E move correspondingly and the display is successively switched over to slice images corresponding to the intersection point OX, such that the display of the slice images as represented by broken lines is switched over to that represented by solid lines. Display of the dipole estimation results 190a is also changed correspondingly to that of the slice images. By the above formation, the positions of signal sources are able to be visually recognized stereoscopically.

That is, in this example, the biological tomographic images include: a first tomographic image that is a cross section in a first direction (for example, the slice image 190A); a second tomographic image that is a cross section in a second direction orthogonal to the first direction (for example, the slice image 190B); and a third tomographic image that is a cross section in a third direction orthogonal to the first direction and second direction (for example, the slice image 190C). When the display control unit 251 receives input for change of the position of a tomographic direction of any one of the first tomographic image, the second tomographic image, and the third tomographic image; the display control unit 251 executes control to switch over to the display of tomographic images in tomographic directions changed correspondingly, for the other tomographic images.

Further, in this embodiment, the control unit 250 receives selection from the strip waveforms parallelly displayed in the area 301A. The display control unit 251 may change the display color or the background color of the selected strip waveform. For example, if the first strip waveform in FIG. 18 is selected, the display of the range represented by the hatched area 108 is changed. If any one of the plural strip waveforms parallelly displayed in the area 301A is selected, the display control unit 251 displays, in the display window 190, the slice images 190A to 190C, which are slice images including a signal source corresponding to the selected strip waveform, and displays information indicating that signal source (the dipole estimation result corresponding to the selected strip waveform), with the information being superimposed on each of the slice images 190A to 190C. If, on the respective slice images 190A to 190C, there is any signal source corresponding to the strip waveforms that have not been selected, information indicating that signal source (dipole estimation result) is also displayed superimposed thereon. On the contrary, if, on the respective slice images 190A to 190C, there are no signal sources corresponding to the strip waveforms that have not been selected, only the information indicating the signal source (dipole estimation result) corresponding to the selected strip waveforms is displayed.

The display control unit 251 is able to display the dipole estimation result corresponding to the selected strip waveform, and the dipole estimation results corresponding to the non-selected strip waveforms, in different display modes, superimposed on the respective slice images 190A to 190C. For example, the dipole estimation result corresponding to the selected strip waveform may be displayed in yellow, and the dipole estimation results corresponding to the non-selected strip waveforms may be displayed in red.

In the example illustrated in FIG. 19, schematically, the selected dipole estimation result is illustrated outlined, and the other dipole estimation results are illustrated in black. When the display at the intersection point O is switched over to the display at the intersection point OX as illustrated in FIG. 20, the dipole estimation results that are displayed in FIG. 19 are no longer displayed, and dipole estimation results corresponding to the respective slice image 190A to 190C in FIG. 20 are displayed. The dipole estimation results in FIG. 19 that are no longer displayed are illustrated in gray in FIG. 20. Thereby, since the dipole estimation result corresponding to the selected strip waveform becomes easy to be visually recognized, positional relations between the signal source corresponding to the selected strip waveform and the other signal sources become easy to be visually recognized, and workability is improved. Further, in this example, without specification of an annotation position of a strip waveform, wherever in the area where the strip waveform is being displayed is selected (which is selectable by a touch operation, clicking with a mouse, or the like), the display is changed to the dipole estimation result corresponding to the singular point of the selected strip waveform. Therefore, operation for switch-over of the dipole estimation results 190a in the display window 190 is facilitated. Further, when any one of the strip waveforms is selected, as illustrated in FIG. 19, by display of the selected strip waveform and the other strip waveforms distinctively from each other, visibility is improved.

By reference back to FIG. 18, explanation will be continued. The display control unit 251 switches over the display of the strip waveforms displayed in the area 301A according to operation on control buttons 340. In this example, there are: a mode where waveforms of all of the sensors are displayed; and a mode where waveforms of arbitrary sensors selected (specified) by the analyst are displayed. In this embodiment, the analyst performs operation of: pressing down a manual button 340B of the control buttons 340; and selecting, with a mouse, a sensor or sensors corresponding to a magnetoencephalography signal waveform or waveforms desired to be displayed in the area 301A, from the magnetoencephalographic distribution diagrams 141 and 142 (clicking one sensor or specifying a range of plural sensors). The display control unit 251 that has received this operation switches over the display in the display areas 101 and 102, so as to display only the signal waveform or waveforms corresponding to the selected sensor or sensors. In association with this switch-over, the display of the dipole estimation results 190a in the display window 190 is also switched over.

For example, if a spike of a different group had occurred at a position of a sensor that was not specified in the magnetoencephalographic distribution diagrams 141 and 142, the dipole estimation result 190a corresponding to that spike is not displayed on the display window 190. Thereby, targets of display are able to be narrowed down to signal sources of a specific range, and thus visibility is improved. For example, by display of only waveforms of magnetoencephalography signals corresponding to sensors around a signal source, from positions of the dipole estimation results 190a on the slice images 190A to 190C and information on the isomagnetic field diagram 150; visual recognition of the magnetoencephalographic waveforms at the same time as the time when the spike was identified by the electroencephalograph is facilitated.

As described above, the analyst verifies reliability of each dipole estimation result 190a being displayed superimposed on the respective slice images 190A to 190C, from positions of the other dipole estimation results 190a, the strip shaped waveforms being displayed in the area 301A, the isomagnetic field diagram 150, and the like, and identifies a position to be removed by surgery (a spot being a cause of epilepsy). If the information processing apparatus 50 receives depression of an output button 140 in the state where the dipole estimation results 190a at the time of that identification are being displayed, the information processing apparatus 50 prints out the screen 400 including: the slice images 190A to 190C, on which the dipole estimation results 190a are displayed superimposed; and the strip waveforms. As described above, stereoscopic positions of the signal sources are able to be identified in more detail than conventionally done.

As described above, in this embodiment, the control to parallelly display, in the area 301A, plural strip waveforms, and display, in the area 301B, the slice images 190A and 190C, on which the signal sources are displayed superimposed, is executed. By this information display control, plural strip waveforms including singular parts are parallelly displayed, and the slice images 190A to 190C, on which the signal sources corresponding to these strip waveforms are displayed superimposed, are also displayed therewith; and thus reliability of identification by the analyst of a target spot being the cause of a case is able to be improved.

Further, as described above, in this embodiment, if any one strip waveform is selected from the plural strip waveforms parallelly displayed, slice images (biological tomographic images) corresponding to the specified strip waveform (including the singular point) are referred to and updated on the same screen. Thus, it becomes easy for the analyst to check slice images of each singular point, and an effect of improving operability and shortening the time period taken by the analyst for analysis is obtained.

Further, for example, when reporting final measurement results, the analyst may extract one or plural noted spots where the most typical waveforms of a singular point or points has/have appeared (for example, characteristic waveforms of epilepsy), from measured signal waveforms for a person to be measured. In that case, by the parallel display of the plural strip waveforms on a single screen, shapes of the waveforms are able to be compared with one another quickly and accurately.

### First Modification of First Embodiment

For example, the display control unit 251 may select annotations indicating an attribute specified at the time of depression of the merging button 300, from plural annotations assigned with the estimation completion marks 182 in FIG. 16, compare plural strip waveforms having one-to-one correspondence to the selected annotations, and group the plural strip waveforms into groups each having the same or similar waveform shapes. For each of the plural groups obtained by this grouping, strip waveforms belonging to that group may be parallelly displayed. If the analyst selects any one of the groups, only strip waveforms belonging to the selected group may be displayed, or if the analysis selects a group desired to be not displayed, strip waveforms belonging to the selected group may be not displayed.

Further, for example, in a case where only one attribute (for example, only “spike”) is expected and specification of an attribute is unnecessary, when the merging button 300 is pressed down, the display control unit 251 may compare plural strip waveform having one-to-one correspondence to plural annotations that have been assigned with the estimation completion marks 82 in FIG. 16, and group the plural strip waveforms into groups each having the same or similar waveform shapes. Furthermore, for example, at the time of analysis, every time signal source estimation is executed for one annotation, the analysis unit 252 may compare strip waveforms corresponding to plural annotations, for which signal source estimation has already been completed, automatically perform grouping of the strip waveforms, and assign, to that one annotation, a group number for identification of the group, together with the estimation completion mark 182. For example, if the display control unit 251 receives depression of the merging button 300 and operation for specification of any one of the group numbers (for example, operation for depression of a button for selection of a group), the display control unit 251 may parallelly display plural strip waveform having one-to-one correspondence to plural annotations belonging to the group indicated by the specified group number, among the plural annotations that have been assigned with the estimation completion marks 182. In this case, on the screen 400 illustrated in FIG. 18, for example, in the display area 310 arranged above the display area 101, in addition to the annotations 110a-1 to 110a-8 displayed at the corresponding time positions on the respective time axes, the group numbers may also be displayed.

Further, in another mode, grouping may be executed by comparison between a waveform (for example, a characteristic waveform of epilepsy) that has been stored in a storage device beforehand (a storage destination for the recording/analysis information storage unit 254, for example) and each of plural strip waveforms having one-to-one correspondence to plural annotations that have been assigned with the estimation completion marks 182 in FIG. 16.

As described above, the timing for the grouping is arbitrary, and thus the grouping may be executed when the merging button 300 is pressed down, or when signal source estimation for one annotation is completed at the time of analysis. Further, the grouping is not necessarily executed fully automatically, and may be executed semi-manually. For example, the analyst may perform an operation that triggers grouping for grouping of plural annotations, for which signal source estimation has been completed, and the grouping may be executed in response to this operation.

In short, by executing, based on similarity among signal waveforms, grouping of plural strip waveforms having one-to-one correspondence to plural annotations, for which signal source estimation has been completed; for each of plural groups obtained by the grouping, the display control unit 251 is also able to parallelly display plural strip waveforms belonging to that group. Further, according to operation by the analyst, only one or more groups of the plural groups may be selectively displayed, or selectively not displayed.

### Second Modification of First Embodiment

When display of respective strip waveforms displayed in the area 301A is switched over according to operation on the control buttons 340, the display control unit 251 may, for example, display dipole estimation results 190a on the slice images 190A to 190C before the switch-over and dipole estimation results 190a thereon after the switch-over, in different colors. Thereby, operability for the analyst is improved, because the analyst is able to visually recognize the estimation results 190a corresponding to the sensors, distinctively.

### Third Modification of First Embodiment

In the above described first embodiment, the strip waveforms and the slice images 190A to 190C are displayed to be capable of being looked through, but not being limited to this display, for example, the area 301A and the area 301B may be displayed on different display devices. By this display of the strip waveforms and the slice images 190A to 190C individually on the different devices, visual recognition of details of the waveforms is facilitated.

### Fourth Modification of First Embodiment

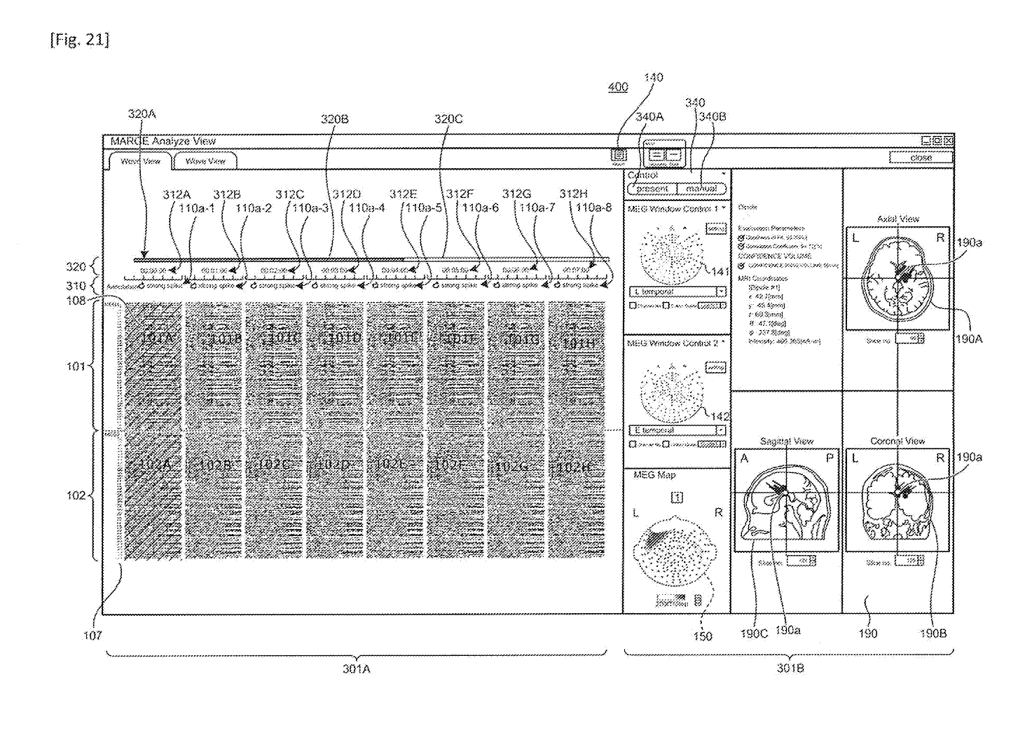

One of more sets of biological signals to be displayed in the above described first embodiment include magnetoencephalography signals acquired from a magnetic sensor group corresponding to the right side of the head of a person to be measured, magnetoencephalography signals acquired from a magnetic sensor group corresponding to the left side of the head of the person to be measured, and electroencephalography signals acquired from electrodes for electroencephalographic measurement of the person to be measured, but not being limited thereto, for example, only the magnetoencephalography signals may be displayed without the display of the electroencephalography signals. In that case, the screen 400 illustrated in FIG. 18 is displayed in a mode illustrated in FIG. 21. In FIG. 21, similarly to FIG. 18, reference signs for display areas representing waveform types of strip waveforms are also assigned in the display areas 101 and 102. The hatched area 108 in FIG. 21 represents the first strip waveform. Each strip waveform is formed of a pair of sets of waveforms displayed in the display areas 101 and 102. For example, the first strip waveform is formed of a pair of: waveforms displayed in the display area 101A; and waveforms displayed in the display area 102A.

### Fifth Modification of First Embodiment

In the above described first embodiment, information on the annotations assigned on the measurement recording screen 201 (FIG. 5 to FIG. 9) is able to be used on the analysis screen 202 (FIG. 11 to FIG. 14). However, not being limited thereto, for example, the recorder may take a note of the times of the singular points of the waveforms at the time of recording (which may be, in a notebook, or with a device having a memoing function), and the analyst may input a time of a singular point on the analysis screen 202 and execute dipole estimation for each of the singular points. In this configuration also, the same effects as those of the above described first embodiment are obtained.

### Sixth Modification of First Embodiment

In the above described first embodiment, information on the annotations assigned on the measurement recording screen 201 (FIG. 5 to FIG. 9) is able to be used on the analysis screen 202 (FIG. 11 to FIG. 14), and the screen 400 illustrated in FIG. 18 is displayed by depression of the merging button 300 after dipole estimation is executed through the analysis screen 202. In this modification, for example, similarly to the above described fifth modification, the recorder takes a note of times of singular points of the waveforms at the time of recording. When the analyst presses down the merging button 300 (which may be a button having another name), a screen, through which times of singular points corresponding to signal sources to be merged are input, is displayed. When the analyst inputs the times on this screen and presses an execution button, the analysis unit 252 estimates signal sources of the singular points corresponding to the input times. The display control unit 251 then executes control to merge the estimated signal sources and display the screen 400 of FIG. 18. By this configuration also, the same effects as those of the above described first embodiment are obtained.

### Second Embodiment

Next, a second embodiment will be described. Description of those in common with the above described first embodiment will be omitted as appropriate. The basic device configuration of this embodiment is the same as that of the above described first embodiment.

The display control unit 251 according to this embodiment calls, based on association information associating between each signal source that has been estimated and signal processing conditions of that estimation, signal processing conditions associated with a signal source corresponding to a strip waveform to be displayed, and reproduces and displays waveforms of the strip waveform at the time of estimation, according to the signal processing conditions called. In this example, signal processing (filtering or the like) aimed for noise reduction has been executed on the biological signals. Further, at the time of signal source estimation, waveforms from sensors other than sensors for detection of biological signals considered to be of a range where a signal source has been generated are not used and not displayed, and waveforms from the sensors corresponding to the signal source are displayed and estimation is executed therefor. In this example, the analyst is able to preform operation for specification of sensors to be used, before pressing down a signal source estimation button 212. Based on signal waveforms from the sensors specified by the analyst, the information processing apparatus 50 (analysis unit 252) executes signal source estimation.

For example, in the area 203A on the analysis screen 202 illustrated in FIG. 14, an arbitrary filter settable by the analyst has been set. When enlarged display is executed in the enlarged display area 200, a filter having the same properties as those of the filter that has been set in the area 203A is able to be applied. However, not being limited thereto, an arbitrary filter different from the filter that has been set in the area 203A may be applied to the enlarged display. In this example, the analysis unit 252 executes dipole estimation, based on signal waveforms being displayed in the enlarged display area 200. When the dipole estimation is executed, the recording/analysis information storage unit 254 stores, on a storage device, association information associating between signal processing conditions and a result of the estimation, the signal processing conditions being: filter conditions applied to the enlarged display area 200; and channel information indicating channels of the signal waveforms being displayed (information indicating the corresponding sensors).

That is, in this example, every time signal source estimation is completed, association information associating between a result of the estimation (the signal source) and signal processing conditions of the estimation (filter conditions and channel information) is stored in the storage device. An estimation result in this example is, not only a dipole estimation result, but also information that enables identification of a strip waveform corresponding thereto (a biological signal group used in the dipole estimation) and an annotation corresponding thereto.

In a case where the display control unit 251 receives depression of the merging button 300 arranged below the annotation list 180 illustrated in FIG. 16, the display control unit 251 calls, based on the above described association information, signal processing conditions associated with the corresponding estimation result for each strip waveform, and reproduces and displays, according to the signal processing conditions called, waveforms of that strip waveform at the time of estimation.

FIG. 22 is a diagram illustrating an example of strip waveforms in the area 301A displayed in this case. In FIG. 22, the reference signs, for the display areas 101A, 102A, 103A, 101B, 102B, . . . displayed in the display areas 101 and 103 are assigned for explanation in the same arrangement as FIG. 18. In this example also, each strip waveform is formed of a set of waveforms displayed in the display areas 101, 102, and 103 as illustrated in FIG. 18. For example, the first strip waveform is formed of a set of: waveforms displayed in the display area 101A; waveforms displayed in the display area 102A; and waveforms displayed in the display area 103A. In this example, the signal waveforms displayed in the display area 101 and the signal waveforms displayed in the display area 102 have been thinned down for display so as to be signal waveforms corresponding to sensors specified as signal processing conditions. For example, if the number of specified sensors corresponding to the display area 101 is greater than the number of specified sensors corresponding to the display area 102, the number of signal waveforms displayed in the display area 101 becomes greater than the number of signal waveforms displayed in the display area 102.

Since specification of sensors for magnetoencephalography signals and specification of sensors for electroencephalography signals are different operations, even if waveforms of magnetoencephalography signals (signal waveforms displayed in the display area 101 and 102) have been thinned down, waveforms of electroencephalography signals (signal waveforms displayed in the display area 103) have not been necessarily thinned down.

Further, if the number of specified sensors corresponding to any one of the three display areas (display areas 101, 102, and 103) forming a strip waveform is “0”, in the display area with the specified number, “0”, no signal waveforms are displayed.

### Modification of Second Embodiment

In the above described second embodiment, when the screen 400 (see FIG. 22) is displayed, as each strip waveform displayed in the area 301A, waveforms of the strip waveform at the time of estimation are reproduced, but limitation is not made thereto. For example, each strip waveform may correspond to all of the sensors, and may be displayed as a biological signal group that has been subjected to default signal processing (see FIG. 18), and depression of a preset button 340A of the control buttons 340 (see FIG. 18) may trigger the display to be switched over to display of waveforms (see FIG. 22) of the strip waveform at the time of estimation. For example, if the display control unit 251 receives depression of the preset button 340A in a state where the screen 400 illustrated in FIG. 18 has been displayed, the display control unit 251 calls, for each of the plural strip waveforms in the area 301A, signal processing conditions associated with the corresponding signal source, and reproduces and displays waveforms of the strip waveform at the time of estimation as illustrated in FIG. 22, according to the signal processing conditions called.

### Third Embodiment

Next, a third embodiment will be described. Description of those in common with the above described embodiments will be omitted, as appropriate. The basic device configuration of this embodiment is the same as that of the above described first embodiment. In each of the above described embodiments, biological data corresponding to a predetermined time period (which may be regarded as “one set of biological data”) are displayed on the analysis screen 202, but in this embodiment, plural sets of biological data that have been divided by a predetermined time period may be a target to be displayed, and signal waveforms of any one of these sets of biological data corresponding to the time zone 120b are displayed.

Further, control to identify, for each of the plural sets of biological data that have been divided by a predetermined time period, one or more biological signal groups corresponding to a part of the biological data, and parallelly display the one or more (one or plural) biological signal groups identified over the plural sets of biological data, is executed. In this example, for each of the plural sets of biological data, the display control unit 251 identifies, among plural annotations that have been input for that set of biological data, plural strip waveforms (biological signal groups) having one-to-one correspondence to annotations, for which signal source estimation has been completed. The display control unit 251 then executes control to parallelly display all of the strip waveforms identified over the plural sets of biological data. That is, the display control unit 251 executes control to parallelly display one or more strip waveforms (partial data) that are over the plural sets of biological data that have been divided by a predetermined time period. Hereinafter, specific content of this control will be described.

### Operation in Measurement Recording

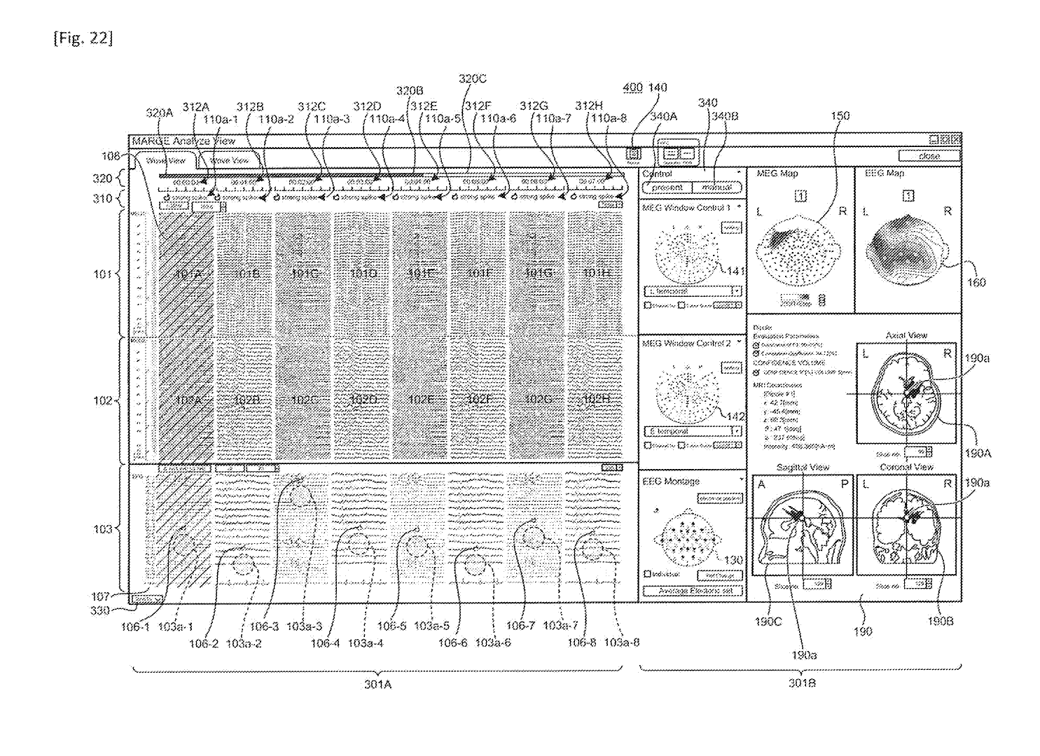

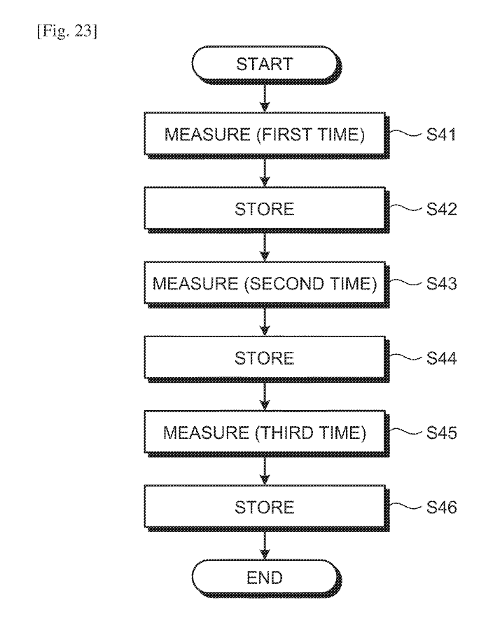

For example, a case where the measurement described in the first embodiment is intermittently executed for three times will now be supposed. It is assumed that a predetermined interval (the time period of the interval being arbitrary) is provided between the measurements. The number, “three times”, is just an example, and is not limited to this example. In short, the number of measurements is arbitrarily changeable according to the aim of the examination. FIG. 23 is a flow chart illustrating an example of operation (operation in measurement recording over three measurements) of the information processing apparatus 50 in this case. As illustrated in FIG. 23, at Step S41, the information processing apparatus 50 executes the first measurement. Operation in the first measurement is the same as the processing of Step S12 to Step S17 in FIG. 10. When the first measurement is ended, the information processing apparatus 50 stores biological data acquired through the first measurement and measurement data including annotations that have been input, in association with a subject ID identifying the subject (Step S42).

Next, the information processing apparatus 50 executes the second measurement (Step S43). The operation in this second measurement is the same as the processing of Step S12 to Step S17 in FIG. 10. When the second measurement is ended, the information processing apparatus 50 stores biological data acquired through the second measurement and measurement data including annotations that have been input, in association with the subject ID (Step S44).

Next, the information processing apparatus 50 executes the third measurement (Step S45). The operation in this third measurement is the same as the processing of Step S12 to Step S17 in FIG. 10. When the third measurement is ended, the information processing apparatus 50 stores biological data acquired through the third measurement and measurement data including annotations that have been input, in association with the subject ID (Step S46).

As described above, every time one measurement (a measurement over a predetermined time period) is completed, measurement data representing results of that measurement are stored in a file unit. In the following description, a stored file of measurement data for one measurement may be referred to as “measurement file”. In this example, after measurement of three times is ended, three measurement files have been stored. Hereinafter, the measurement file corresponding to the first measurement may be referred to as “first measurement file”, the measurement file corresponding to the second measurement as “second measurement file”, and the measurement file corresponding to the third measurement as “third measurement file”. As described above, each of the measurement files is stored in association with the subject ID.

### Operation in Analysis

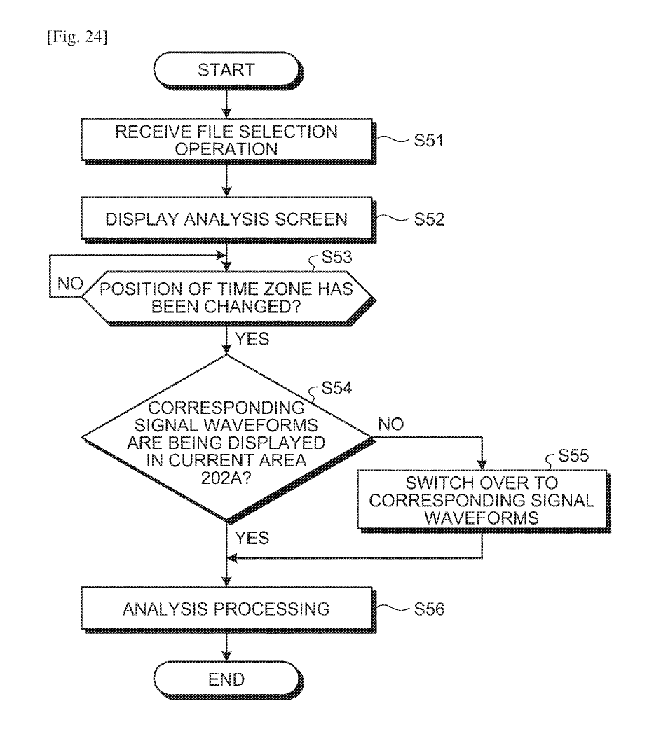

Next, operation in analysis will be described. It is supposed herein that if depression of the “Analysis” button 204-2 is received through the start screen 204 of FIG. 4, the information processing apparatus 50 (display control unit 251) displays a selection screen for selection of a measurement file acquired by measurement, on the display device 28. FIG. 24 is a flow chart illustrating an example of operation by the information processing apparatus 50 in this case.

Firstly, the information processing apparatus 50 (control unit 250) receives an operation for selection of a measurement file through the selection screen (Step S51). Next, the information processing apparatus 50 (control unit 250) executes control to: read a series of measurement files (the above described three measurement files in this example) including the measurement file selected in Step S51, and one or more other measurement files associated with the same subject ID as the subject ID associated with the selected measurement file, and displaying the analysis screen 202 reflecting that read series of measurement files, on the display device 28 (Step S52).

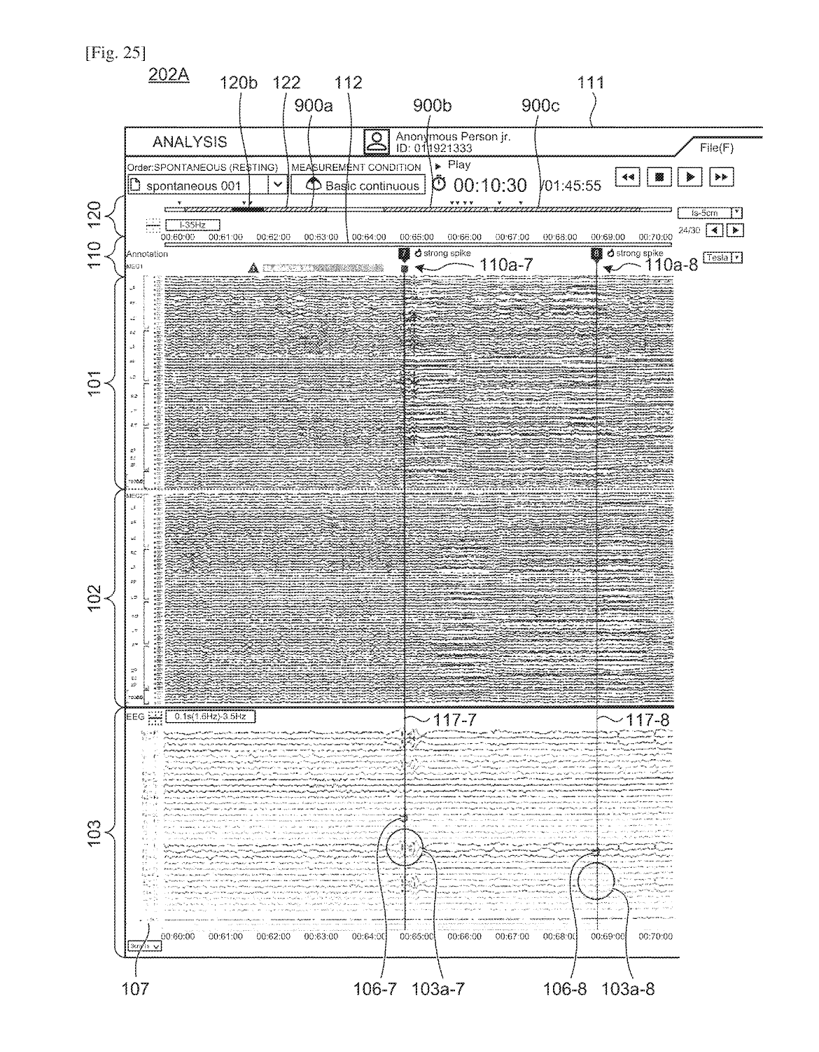

FIG. 25 is a diagram illustrating an example of the area 202A at the left side of the analysis screen 202 at that time. On the time axis 122, instead of the overall recording time period corresponding to any one of the measurement files, the overall time period is displayed, the overall time period including all of recording time periods of the series of measurement files (the first measurement file, the second measurement file, and the third measurement file). on the time axis 122, range information 900a indicating the recording time period of the first measurement file, range information 900b indicating the recording time period of the second measurement file, and range information 900c indicating the recording time period of the third measurement file are displayed. In the following description, when these pieces of range information 900a, 900b, and 900c are distinguished from one another, they may each be simply referred to as “range information 900”. Each piece of range information 900 may be added with information indicating the name of the corresponding measurement file. In this example, since the measurements are carried out with intervals therebetween, on the time axis 122, gaps (blank areas) are provided between the pieces of range information 900.

By performing an operation of moving the time zone 120b with a mouse or the like, the analyst is able to switch over the signal waveforms displayed in the area 202A. In this example, the signal waveforms corresponding to the time zone 120b (a part of biological data of any one of the measurement files) are displayed in the area 202A. That is, by moving the time zone 120b on the time axis 122, the analyst is able to display signal waveforms of a desired time zone across a measurement file, in the area 202A. Further, in the annotation list 180 in the area 202B at the right side of this analysis screen 202, all of annotations included in each of the three measurement files are displayed. Further, for example, each of the measurement files may be associated with the name of the corresponding examination, and the name of examination corresponding to the time zone 120b may be displayed together on the analysis screen 202.

By reference back to FIG. 24, explanation will be continued. If, after displaying the analysis screen 202 in Step S52, the information processing apparatus 50 (control unit 250) receives an operation for change of the position of the time zone 120b (YES at Step S53), the information processing apparatus 50 checks whether or not signal waveforms corresponding to the changed position of the time zone 120b are currently being displayed in the area 202A (Step S54).

If a result of Step S54 is NO (No at Step S54), the information processing apparatus 50 (control unit 250) switches over the signal waveforms displayed in the area 202A to signal waveforms corresponding to the changed position of the time zone 120b (Step S55). If a result of Step S54 is YES (YES at Step S54) or after Step S55, the information processing apparatus 50 (control unit 250) executes analysis processing according to operation by the analyst (Step S56). The content of this analysis processing corresponds to the processing of Step S23 to Step S31 illustrated in FIG. 17.

As described above, in the annotation list 180, all of the annotations included in each of the three measurement files are displayed. If depression of the merging button 300 is received at Step S29, the display control unit 251 displays the screen 400, displays, in the area 301A, a strip waveform of each of plural annotations that have been assigned with the estimation completion marks 182, among the plural annotations displayed in the annotation list 180 (all of annotations over the plural measurement files), and displays, in the area 301B, analysis information on the strip waveforms displayed in the area 301A. The rest of the operation is the same as that described above with respect to the first embodiment.

A modification for grouping may also be considered similarly to the above described first embodiment. For example, the display control unit 251 may select annotations indicating an attribute also specified at the time of depression of the merging button 300, from plural annotations that have been assigned with the estimation completion marks 182, compare plural strip waveforms having one-to-one correspondence to the selected annotations, and group the plural strip waveforms into strip waveform groups each having the same or similar waveform shapes. For each of the plural groups obtained by this grouping, strip waveforms belonging to that group may be parallelly displayed. In this case, each group includes one or more strip waveforms corresponding to one or more measurement files.

For example, a group: may include one or more strip waveforms corresponding to only one of the measurement files; may include one or more strip waveforms corresponding to the first measurement file and one or more strip waveforms corresponding to the second measurement file (or third measurement file); or may include one or more strip waveforms corresponding to the first measurement file, one or more strip waveforms corresponding to the second measurement file, and one or more strip waveforms corresponding to the third measurement file. If the analyst selects any one of the groups, only strip waveforms belong to the selected group may be displayed, or if the analyst selects a group desired to be not displayed, strip waveforms belonging to the selected group may be not displayed.

Further, for example, in a case where only one attribute (for example, only “spike”) is expected and specification of an attribute is unnecessary, when the merging button 300 is pressed down, the display control unit 251 may compare plural strip waveforms having one-to-one correspondence to plural annotations that have been assigned with the estimation completion marks 182 (plural annotations over the plural measurement files), and group the plural strip waveforms into groups each having the same or similar waveform shapes. Furthermore, for example, at the time of analysis, every time signal source estimation is executed for one annotation of one measurement file; the analysis unit 252 may: compare strip waveforms corresponding to plural annotations, for which signal source estimation has already been completed (plural annotations over the plural measurement files); automatically perform grouping of the strip waveforms; and assign, to that one annotation, a group number for identification of the group, together with the estimation completion mark 182. For example, if the display control unit 251 receives depression of the merging button 300 and operation for specification of any one of the group numbers, the display control unit 251 may parallelly display plural strip waveforms having one-to-one correspondence to plural annotations belonging to the group indicated by the specified group number, among plural annotations that have been added with the estimation completion marks 182 (plural annotations over the plural measurement files).

Further, in another mode, grouping may be executed by comparison between a waveform (for example, a characteristic waveform of epilepsy) that has been stored in a storage device beforehand (the recording/analysis information storage unit 254, for example) and each of plural strip waveforms having one-to-one correspondence to plural annotations that have been assigned with the estimation completion marks 182 (plural annotations over the plural measurement files).

### First Modification of Third Embodiment

For example, on the time axis 122 on the analysis screen 202, only the range information corresponding to any one of the measurement files may be displayed, and according to operation by the analyst, the range information 900 on the time axis 122 may be switched over in measurement file units. FIG. 26 is a diagram illustrating an example of the analysis screen 202. In the example of FIG. 26, on the time axis 122, only the range information 900a indicating the recording time period of the first measurement file is displayed. If the information processing apparatus 50 (display control unit 251) receives an operation for switch-over of the range information 900 by the analyst, the information processing apparatus 50 switches over the range information 900 on the time axis 122 in measurement file units according to the received operation, and switches over the display in the area 202A and the area 202B correspondingly to the measurement file after the switch-over.

### Second Modification of Third Embodiment

In the above described third embodiment, the position of the time zone 120b is set so as to not extend over different pieces of range information 900. For example, if an operation for advancement of the position of the time zone 120b by one step is received in a state where the time zone 120b is positioned at the end point of the range information 900a illustrated in FIG. 25, the information processing apparatus 50 (display control unit 251) switches over the display of the time zone 120b such that the time zone 120b is positioned at the start point of the next range information 900b without extending over both the range information 900a and the range information 900b.

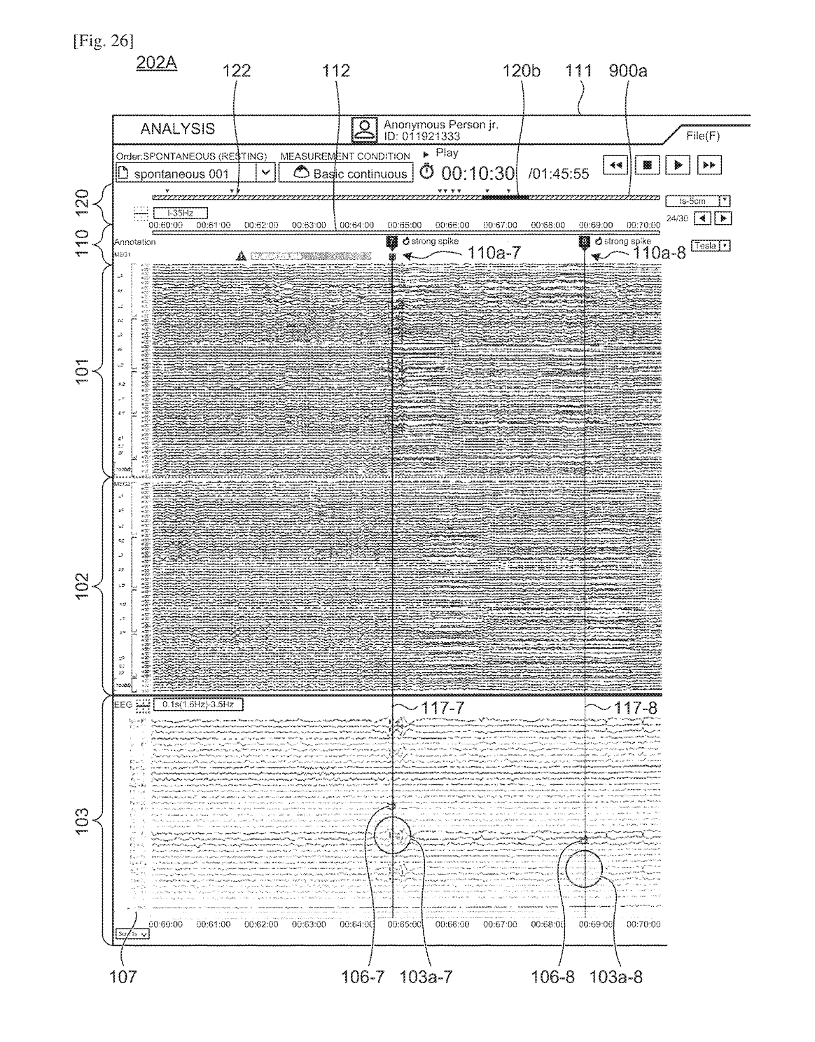

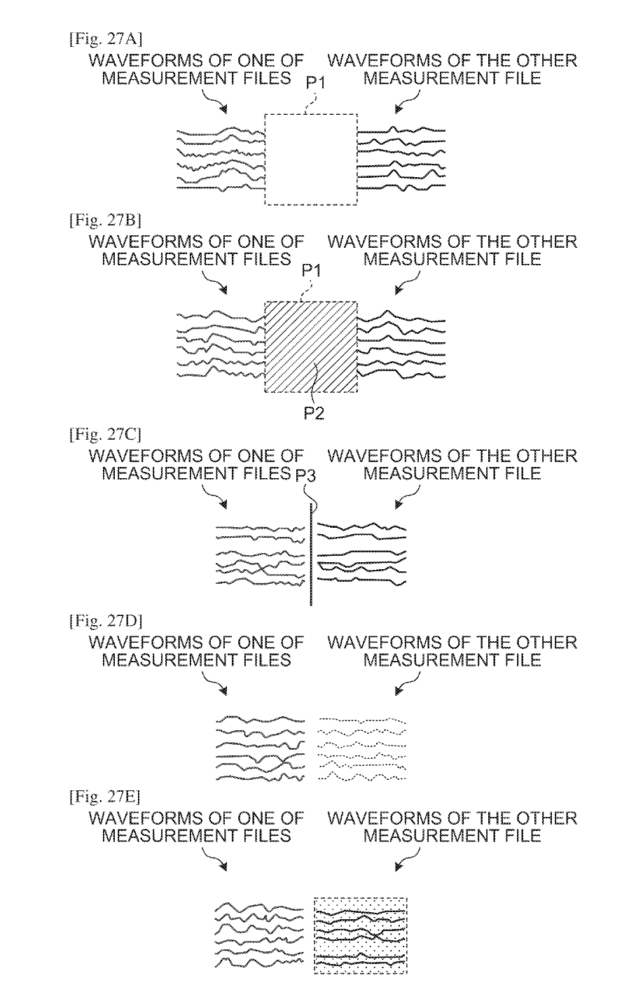

However, not being limited thereto, in this modification, the time zone 120b may be arranged to extend over different pieces of range information 900. In this case, as illustrated in FIG. 27A, a blank area P1 (corresponding to a gap between measurements) where no biological signals exist is generated in signal waveforms corresponding to the time zone 120b. In order to make it easy for the blank area P1 to be visually recognized as an area between different measurement files, as illustrated in FIG. 27B, the information processing apparatus 50 (display control unit 251) may change the background of the blank area P1 to be in a display mode P2, in which the background of the blank area P1 is easy to be visually recognized. Herein, as an example of the display mode P2, a hatched display mode is illustrated, but as long as the display mode P2 is a display mode that facilitates visual recognition, any display mode may be adopted. For example, the background color may be changed to a conspicuous color, or the hatch may be changed to a different pattern.

Further, for example, it is supposed that if the time zone 120b is arranged to extend over different pieces of range information 900 and the time interval between the measurements is short, there is hardly any gap between the signal waveforms corresponding to one of the measurement files and the signal waveforms corresponding to the other one of the measurement files. In this case, as illustrated in FIG. 27C, for example, the information processing apparatus 50 (display control unit 251) may display a line P3 (a line different from the annotation line) indicating a joint between the signal waveforms corresponding to one of the measurement files and the signal waveforms corresponding to the other one of the measurement files.

Further, as illustrated in FIG. 27D, signal waveforms corresponding to one of the measurement files and signal waveforms corresponding to the other one of the measurement files may be displayed differently with different colors or densities, or as illustrated in FIG. 27E, for example, the background color of signal waveforms corresponding to any one of the measurement files may be changed.

### Third Modification of Third Embodiment

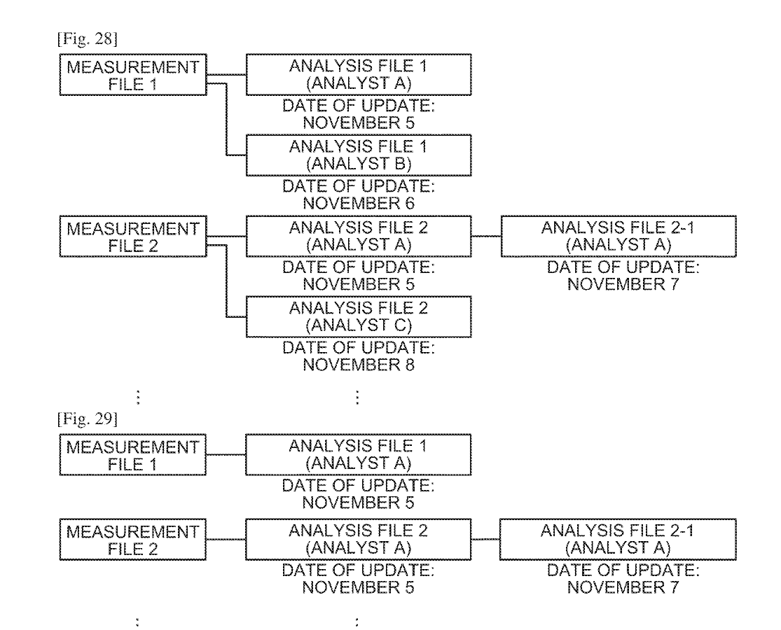

As illustrated in FIG. 28, the information processing apparatus 50 according to the third embodiment manages (stores) each of plural measurement files in association with an analysis file representing results of analysis (analysis information) thereon. The number of analysts is not limited to one, and a case where each of plural analysts carries out analysis may also be supposed. In this case, a different analysis file is generated for each of the plural analysts, and is associated with a measurement file. Further, in this example, every time analysis is ended, an analysis file representing results of that analysis is newly associated with a measurement file. That is, when the information processing apparatus 50 receives input of an analysis ending command every time analysis of one time is executed, the information processing apparatus 50 stores an analysis file representing results of that analysis newly in association with a measurement file. For example, depression of a button indicating “storage” or “end” on the analysis screen 202 by an analyst may trigger the input of an analysis ending command.

When annotations of all of the files are displayed in the annotation list 180 on the analysis screen 202, an analyst currently logged in and performing analysis is presented with also annotations, on which the analyst is not performing the analysis, and since the analyst needs to search for annotations analyzed by the analyst from all of the annotations, this need is a big burden on the analyst.

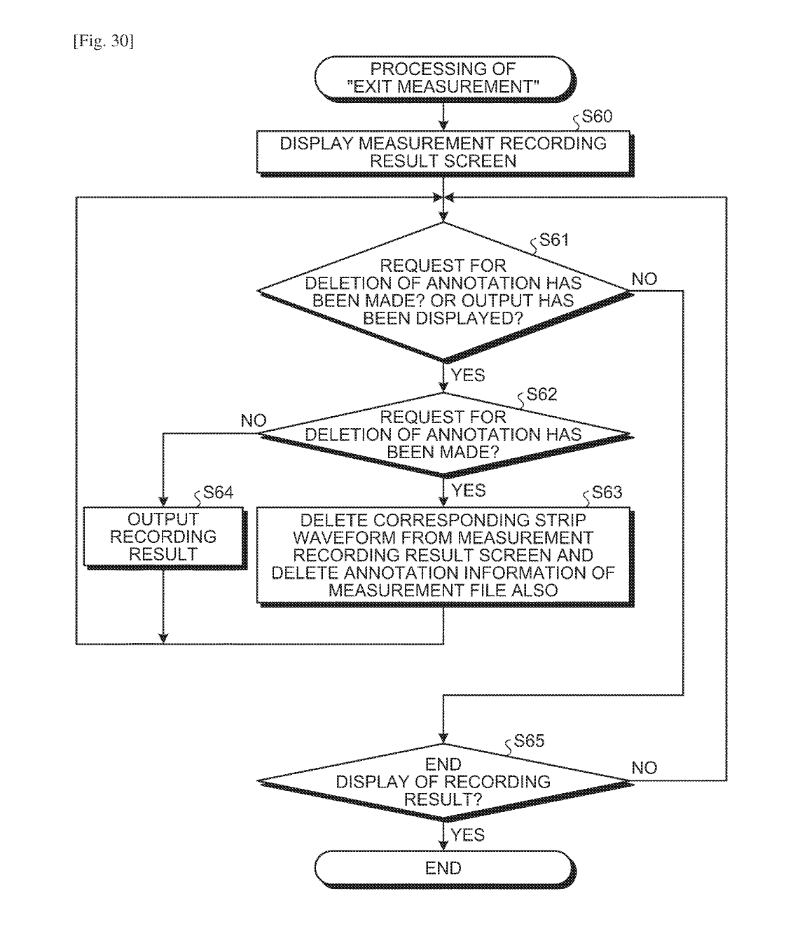

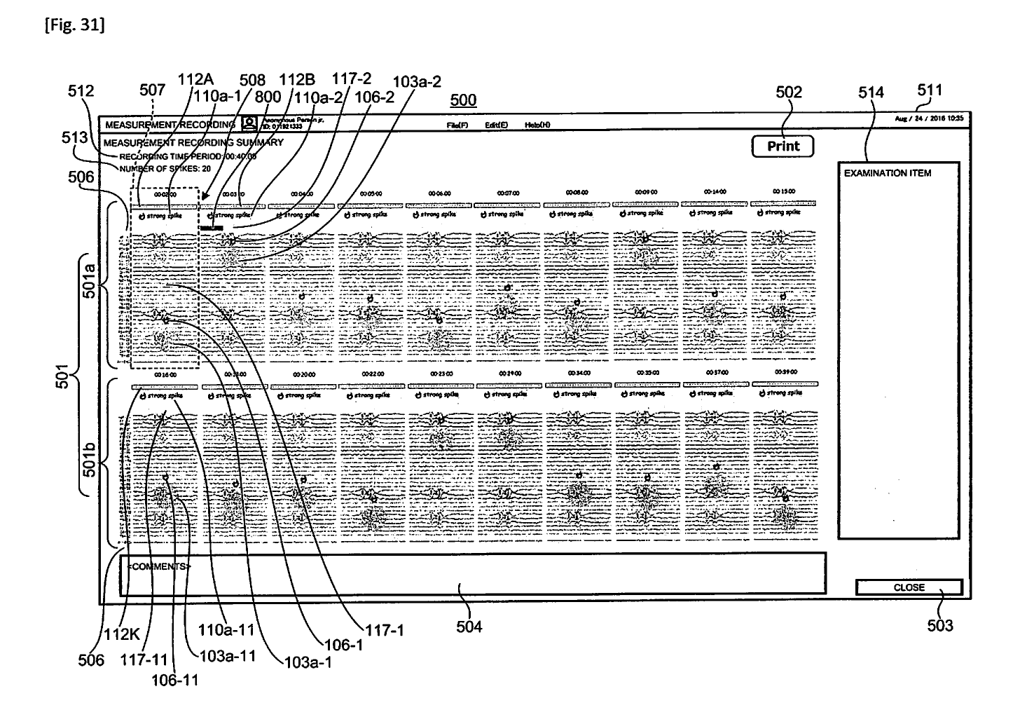

Therefore, in this modification, the information processing apparatus 50 manages each analysis file in association with the analyst, the date of update (the date of generation of the analysis file), and the subject ID. When the information processing apparatus 50 (display control unit 251) receives depression of the “Analysis” button 204-2 through the start screen 204 in FIG. 4, the information processing apparatus 50 extracts, among analysis files included in a file list as illustrated in FIG. 28, analysis files corresponding to the analyst who is currently logged in, and displays, on a selection screen of the display device 28, options having the extracted analysis files that are associated with plural measurement files. For example, if the analyst who is currently logged in is an analyst “A”, from the analysis files included in the file list in FIG. 28, analysis files corresponding to the analyst “A” are extracted associatively as illustrated in FIG. 29, the extracted analysis files are hierarchically displayed on the selection screen, and a predetermined file is received by selection. As to any measurement file not associated with any of the extracted analysis files, only that measurement file is received.

If, for example, the information processing apparatus 50 receives selection of any one of the measurement files through this selection screen, the information processing apparatus 50 displays the analysis screen 202 reflecting a series of measurement files including: the selected measurement file; and one or more other analysis files associated with the same subject ID as the subject ID associated with the selected measurement file. Upon display of the annotation list 180 on this analysis screen 202, for each of a series of measurement files, the information processing apparatus 50 (display control unit 251) identifies an analysis file associated with that measurement file, and displays annotations corresponding to the identified analysis file in the annotation list 180. Further, if plural analysis files of the same analyst have been associated with a single measurement file, only the analysis file of the latest data of update is identified, and annotations corresponding to the identified analysis file are displayed in the annotation list 180. Further, if an analysis file has not been associated with a measurement file, annotations included in that measurement file are displayed in the annotation list 180.

Further, for example, an analyst may select all of measurement files desired to be displayed, on the selection screen. For example, if, on the selection screen, the analyst “A” selects a measurement file 2, the information processing apparatus 50 (display control unit 251) may display the analysis screen 202 reflecting only the measurement file 2. For example, upon display of the annotation list 180 on this analysis screen 202, the information processing apparatus 50 (display control unit 251) displays annotations corresponding to an analysis file 2-1 with the latest date of update, among two analysis files 2 and 2-1 associated with the measurement file 2, in the annotation list 180.

Accordingly, since only annotations corresponding to the analyst who is currently logged in are appropriately displayed in the annotation list 180 on the analysis screen 202, convenience for the analyst is improved.

Being logged in means having authorization to use the information processing apparatus 50, and the information processing apparatus 50 has a function of determining possibility of log-in of a user (analyst). For example, at the time of starting of the information processing apparatus 50, a log-in screen that prompts input of information for log-in (for example, log-in information formed of a combination of an ID and a password) is displayed, and the analyst inputs, through the log-in screen, log-in information that the analyst possesses. For each of users having authorization to use the information processing apparatus 50, the information processing apparatus 50 registers preset log-in information in association with the user; and if log-in information input through the log-in screen matches the log-in information that has been registered, the information processing apparatus 50 permits log-in of the user (analyst) who has input the log-in information, and if the log-in information input through the log-in screen does not match the log-in information that has been registered, the information processing apparatus 50 does not permit the log-in.

### Fourth Embodiment

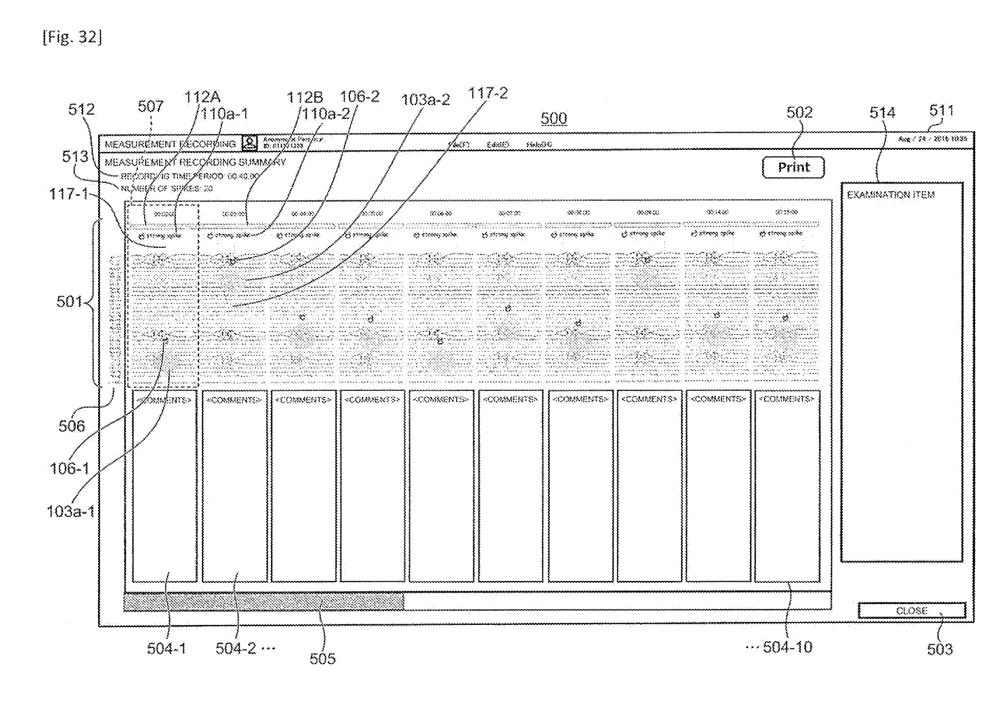

Next, by use of FIG. 30 and FIG. 31, a fourth embodiment will be described. In this fourth embodiment, a mode, in which strip waveforms, which are a list of waveforms at positions that have been assigned with annotations at the time of measurement recording, are parallelly displayed, and processing, such as correction, is executed at the measurement recording stage, will be described.

FIG. 30 is a flow chart illustrating an example of information display processing at a measurement recording stage according to the fourth embodiment. FIG. 30 illustrates processing continued from Step S17 illustrated in FIG. 10.

In this example, by depression of the “Exit Measurement” button 179 illustrated in FIG. 9, the information processing apparatus 50 (control unit 250) displays a measurement recording result screen (Step S60), which will be described later.

The measurer is able to check the measurement recording result screen, and perform editing operation on recording results and output operation on the recording results. The information processing apparatus 50 (control unit 250) determines which one of a request for deletion of an annotation, a request for output of a screen, or a request for ending of display of recording results has been made (Step S61, Step S62, and Step S65), and executes processing according to a result of the determination.

For example, it is supposed that an operation for deletion of an annotation assigned to later described recorded waveforms has been made. In this case, the information processing apparatus 50 (control unit 250) determines that deletion of an annotation has been requested (YES at Step S61 and YES at Step S62), deletes the corresponding waveforms from the measurement recording result screen, and deletes or does not display information on the corresponding annotation in the measurement file (Step S63). The information processing apparatus 50 (control unit 250) returns to Step S61, and executes processing according to operation received subsequently.

Further, if the information processing apparatus 50 (control unit 250) receives depression of an output button 502 on the measurement recording result screen (for example, see FIG. 31) (YES at Step S61, and NO at Step S62), the information processing apparatus 50 outputs the information displayed on the measurement recording result screen to a sheet of paper, an electronic medium, an output terminal, or the like (Step S64). The information processing apparatus 50 (control unit 250) then returns to Step S61, and executes processing according to operation received subsequently.

Further, if depression of a button 503 for closure of the window (see, for example, FIG. 31) on the measurement recording result screen is received (NO at Step S61, and YES at Step S65), the measurement recording result screen is closed, and the flow is ended.

### Example of Measurement Recording Result Screen According to Fourth Embodiment: List of all Strip Waveforms

FIG. 31 is an example of the measurement recording result screen displayed at Step S60 in FIG. 30. A measurement recording result screen 500 is a screen, on which a recording date 511, a recording time period (corresponding to “measurement time period”) 512, the number of spikes 513, a list 501 of waveforms at positions where annotations have been specified, an examination name 514, an annotation comment field 504, and the like are displayed to be capable of being looked through. These pieces of information are the display information on the measurement recording screen 201 illustrated in FIG. 5 to FIG. 9 or information accumulated at the time of measurement recording. The information processing apparatus 50 (control unit 250) reads these pieces of information via the recording/analysis information storage unit 254 (see FIG. 3), and displays them on the measurement recording result screen 500.

The numerical value displayed as the number of spikes 513 is the cumulative number in the counter box 118 (for example, see FIG. 6) when the “Exit Measurement” button 179 is pressed down after measurement recording.

In the waveform list 501, plural strip waveforms are parallelly displayed, each of the plural strip waveforms being waveforms of two seconds around a spot assigned with an annotation, the waveforms being among biological data that have been measured and recorded. The strip waveforms will be described by use of reference signs assigned to a strip waveform added with a broken lined frame 507 and a strip waveform adjacent thereto. The other strip waveforms parallelly displayed in the waveform list 501 include, similarly to the strip waveform with the broken lined frame 507, marks for emphasized display, but reference signs therefor are omitted for intelligibility of the display state.

FIG. 31 illustrates the display state in a case where units of the strip waveforms are all signal waveforms of electroencephalography signals as illustrated with the broken lined frame 507. Between adjacent strip waveforms at each stage of the parallel strip waveforms, an interval 508 is provided so that a break therebetween is known.

In the whole waveform list 501, the same number of (twenty, in this example) strip waveforms as the cumulative number indicated by the number of spikes 513 are displayed, and these strip waveforms are arranged in order of recording time. In this example, the strip waveforms are arranged in order of recording time from left to right in a display area 501a at an upper stage, and subsequently from left to right in a display area 501b at a lower stage. For each of the strip waveforms, in addition to the waveforms, a set of: a mark (mark 103a-1, mark 103a-2, . . . , mark 103a-11, . . . ); an attribute icon (attribute icon 106-1, attribute icon 106-2, . . . , attribute icon 106-11, . . . ); a line (line 117-1, line 117-2, . . . , line 117-11, . . . ); a time axis (time axis 112A, time axis 112B, . . . , time axis 112K, . . . ); an annotation indicating a specification result (annotation 110a-1, annotation 110a-2, . . . , annotation 110a-11, . . . ); and the like, is displayed. In addition, the warning display 800 for notification of a strip waveform subjected to warning is also displayed. In this example, the warning display 800 is displayed for the second strip waveform from the left in the display area 501a at the upper stage.

In the comment field 504, text information input in the input box 115b illustrated in FIG. 6 or in a memo field 116 illustrated in FIG. 7 is displayed. Since the waveforms are displayed with text, in association with the annotation numbers of the waveforms, the measurer is able to understand and check reliability of the annotation by collation with the waveforms.

The measurer is able to check whether the strip waveforms are waveforms usable in the next analysis. For example, if the measurer determines, from the warning display 800 and the comment field 504, that the second strip waveform from the left in the display area 501a at the upper stage is difficult to be used in analysis, the annotation corresponding to that strip waveform is able to be deleted. If the annotation is deleted, annotation information corresponding to the waveform is also deleted from the recording information of the measurement file.

According to this example, since reliability of measurement results is able to be checked after the measurement is ended, unfavorable information is able to be deleted at that stage, and determination of whether additional measurement is necessary is possible. Thereby, primary selection of information is possible before analysis, and the identification time period for a target spot being a cause of a case is able to be shortened in the analysis; and thereby also, reliability of identification of the target spot is able to be increased.

### First Modification of Measurement Recording Result Screen According to Fourth Embodiment: Browsing of Strip Waveforms by Scrolling

FIG. 32 is a first modification of the measurement recording result screen 500. The number of strip waveforms displayed in FIG. 31 is the same as the number of spikes 513, “20”, but in this modification, as illustrated in FIG. 32, the number of strip waveforms displayed is the number of strip waveforms that are displayable in one row of the display area 501. Below the strip waveforms, the comment fields 504 (comment field 504-1, comment field 504-2, . . . , comment field 504-10) are displayed, such that comments corresponding to the strip waveforms are able to be checked. Thereby, the ease of looking through is improved. For strip waveforms and comments not being displayed, a scroll bar 505 provided below the comment fields 504 is operated from side to side. At the time of initial display of the measurement recording result screen 500, a strip waveform having the first annotation in the recording time order is displayed at the leftmost side on the screen 500. When the scroll bar 505 is operated, the strip waveforms and the comment fields move with the scroll bar 505 in the operated direction, and when the scroll bar 505 reaches the right end of the screen 500, a strip waveform having the last annotation in the recording time order appears at the rightmost side on the screen 500.

### Second Modification of Measurement Recording Result Screen According to Fourth Embodiment: Display of Only Channels Around Specified Marks

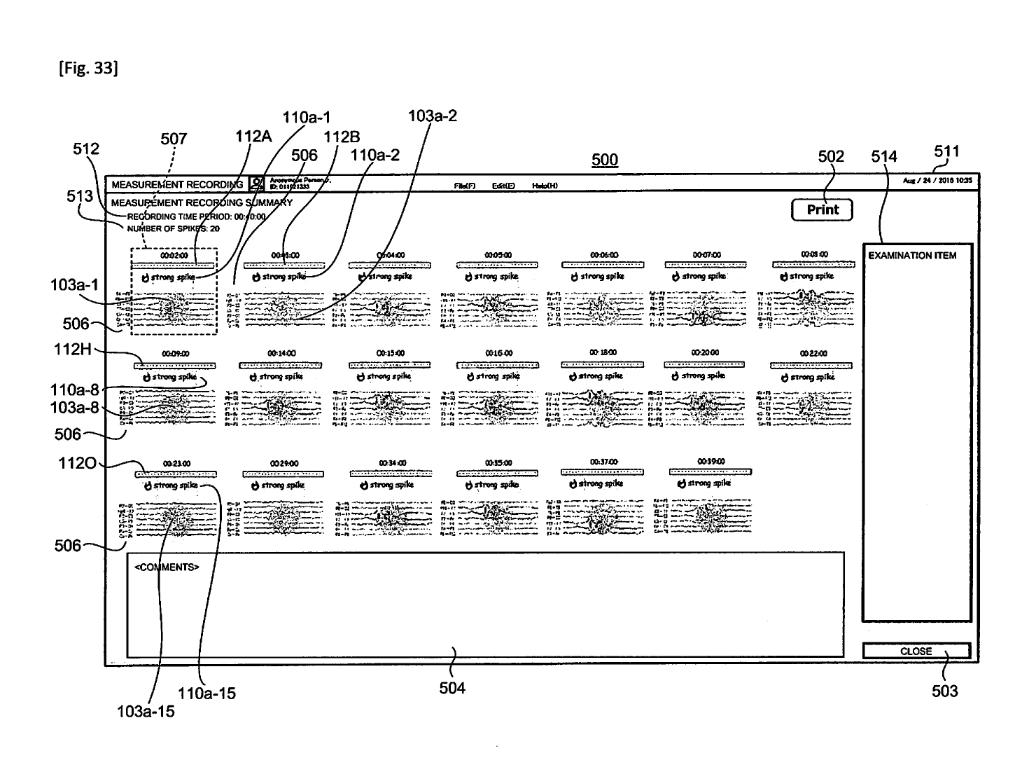

FIG. 33 is another modification of the measurement recording result screen 500. In this modification, the number of strip waveforms displayed is, similarly to FIG. 31, the number of spikes 513, “20”, but it is different from FIG. 31 in that only waveforms corresponding to display ranges of marks specified in the measurement (mark 103a-1, mark 103a-2, . . . ) are displayed. Further, beside each strip waveform, information (channels) 506 indicating a reference electrode and an active electrode for each signal waveform is displayed. This is because positions of the marks (mark 103a-1, mark 103a-2, . . . ) in the channel ranges are not necessarily the same for all of the strip waveforms. In the example illustrated in FIG. 33, all of the twenty strip waveforms are displayed in three stages. In this example, the strip waveforms are parallelly displayed in order of recording time, from left to right at the first stage, subsequently from left to right at the second stage, and lastly from left to right at the third stage.

According to this modification, since waveforms of a noted spot are able to be displayed largely for each strip waveform, visibility of shapes of waveforms in the amplitude direction is particularly improved.

This modification may further be modified to a mode where the strip waveforms are moved by the scroll bar 505 as illustrated in FIG. 32. In this case, the display size of the strip waveforms is able to be increased even more and visibility is improved further.

### Third Modification of Measurement Recording Result Screen According to Fourth Embodiment: Display of Only MEG Waveforms

Since the fourth embodiment and the first modification and second modification of the fourth embodiment are each an example of the case where a measurer specifies a disturbance in waveforms or a singular point of amplitude in signal waveforms of electroencephalography signals, display examples for strip waveforms of electroencephalography signals have been described. However, a case where signal waveforms of magnetoencephalography signals are noted and specified may also be considered. Therefore, modification to display of signal waveforms of magnetoencephalography signals of a right side of a head and signal waveforms of magnetoencephalography signals of a left side of the head, or modification to display enabling switch-over between these sets of signal waveforms may be made.

### Fourth Modification of Measurement Recording Result Screen According to Fourth Embodiment: Display of MEG and EEG Waveforms

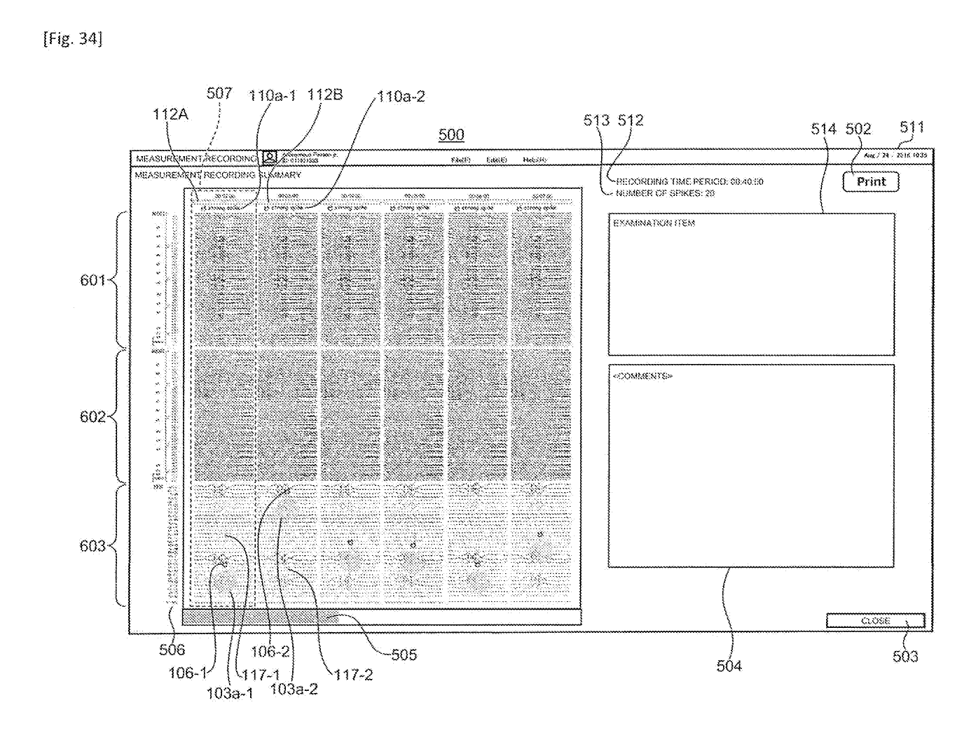

FIG. 34 is a fourth modification of the measurement recording result screen 500. On the measurement recording result screen 500 in FIG. 34, strip waveforms including MEG and EEG waveforms are parallelly displayed. Since the signal waveforms oscillate in an up-down direction of the screen 500, a single signal waveform occupies a vertical width of the screen 500. This is evident also from the measurement recording screen 201 illustrated in FIG. 5, for example. Therefore, in this fourth modification, by arrangement of the recording time period 512 and the number of spikes 513 illustrated in FIG. 31, in the right side area of the screen 500; an area at a left side of the screen 500 is vertically largely obtained, the area being where the strip waveforms are displayed. As indicated by the broken lined frame 507 in FIG. 34, each strip waveform is formed of waveforms of magnetoencephalography signals of the right brain illustrated at the top, waveforms of magnetoencephalography signals of the left brain illustrated in the middle, and waveforms of electroencephalography signals illustrated at the bottom. In the left side display area of the screen 500, the waveforms of the magnetoencephalography signals of the right brain of the respective strip waveforms are parallelly displayed in a display area 601, the waveforms of the magnetoencephalography signals of the left brain of the respective strip waveforms are parallelly displayed in a display area 602, and the waveforms of the electroencephalography signals of the respective strip waveforms are parallelly displayed in a display area 603. That is, on the measurement recording result screen 500, the strip waveforms are parallelly displayed at the left side, and results for the strip waveforms are displayed at the right side. The types of signal waveforms displayed in the respective display areas 601, 602, and 603 are just an example, and they may be displayed by being switched over with one another, as appropriate. For example, the signal waveforms of the right brain may be displayed in the display area 602, and the signal waveforms of the left brain may be displayed in the display area 601 by switch-over between display destinations of the signal waveforms of the right brain and signal waveforms of the left brain.

When the number of strip waveforms displayed on a single screen is the same as the number of spikes, visibility of shapes of the waveforms is degraded depending on the number of spikes. Therefore, in this modification, the scroll bar 505 is provided, and by a leftward or rightward operation on the scroll bar 505, a part of the whole strip waveforms is displayed.

According to the first to third modifications of the fourth embodiment, there is a need for switch-over, according to waveforms to be focused on by a measurer, to only signal waveforms of electroencephalography signals or to only signal waveforms of magnetoencephalography signals, but according to this modification, there is no need for consideration of which waveforms are to be focused on, and thus operability is improved. Further, even if either electroencephalography signals or magnetoencephalography signals have been specified with a mark (mark 103a-1, mark 103a-2, . . . ), since the other waveforms that have not been specified are able to be checked simultaneously on the measurement recording result screen 500, whether the waveforms are usable in analysis is able to be determined from both of these waveforms.

### Fifth Modification of Measurement Recording Result Screen According to Fourth Embodiment: EEG+MRI Images

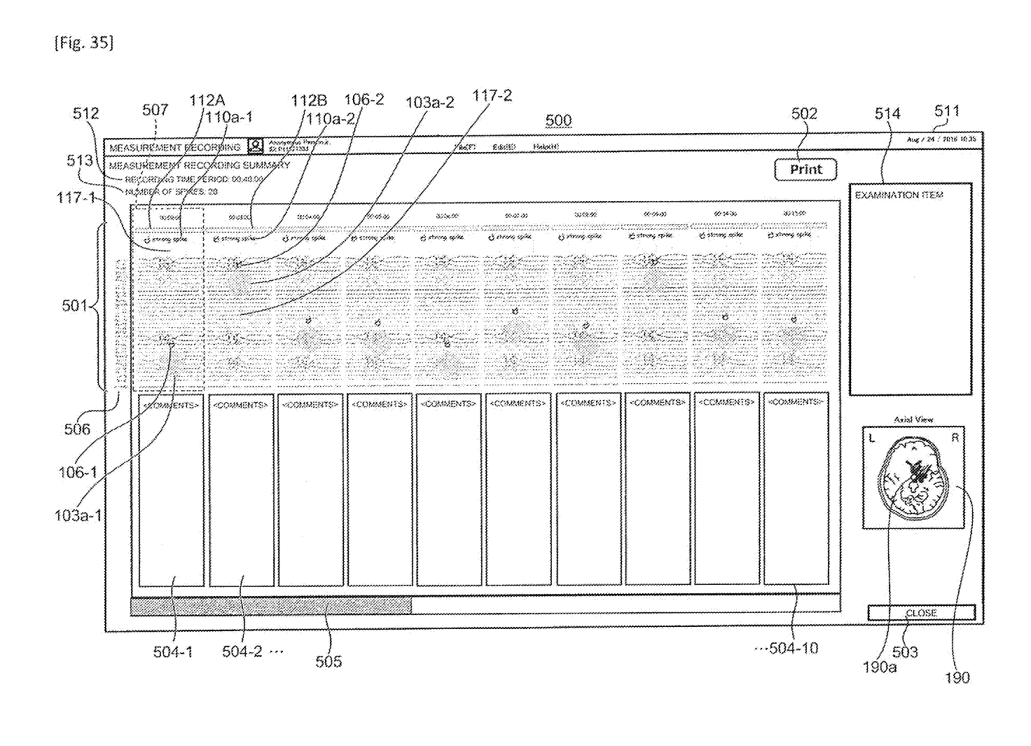

FIG. 35 is a fifth modification of the measurement recording result screen 500. The measurement recording result screen 500 illustrated in FIG. 35 is different from that in FIG. 32 in that the display window 190 for tomographic images is displayed at the right side of the screen 500.

In this fifth modification, when the information processing apparatus 50 (control unit 250) receives depression of the “Exit Measurement” button 179 illustrated in FIG. 9 (YES at Step S17 in FIG. 30), the analysis unit 252 executes calculation for signal source estimation. The estimation result 190a is displayed superimposed in the display window 190. In the display window 190, signal source estimation results corresponding to all of the strip waveforms are displayed. Further, if a strip waveform is specified by a mouse or the like, that strip waveform is displayed distinctively from the other strip waveforms (emphasized display like highlighting), and the corresponding estimation result 190a on the display window 190 is displayed with emphasis.

According to this modification, in consideration of not only shapes of waveforms including a specified spot, but also the position of the signal source, whether the specified spot is usable in analysis is able to be determined.

### Sixth Modification of Measurement Recording Result Screen According to Fourth Embodiment: Summary List of all Measurement Files

FIG. 23 illustrates the flow, in which, in a case where measurements are intermittently performed more than once (three times in that example), each of the “first measurement file” that is a measurement file corresponding to the first measurement, the “second measurement file” that is a measurement file corresponding to the second measurement, and the “third measurement file” that is a measurement file corresponding to the third measurement is stored. Modification may be made such that a measurement recording result screen corresponding to each of the “first measurement file”, “second measurement file”, and “third measurement file” generated in this processing is displayed, and information obtained through this measurement recording result screen is stored by the recording/analysis information storage unit 254 into a storage destination.

Further, modification may be made such that a measurement recording result screen for a combination of measurement files is displayed, the combination including the “first measurement file” to the “third measurement file”, and information obtained through this measurement recording result screen is stored by the recording/analysis information storage unit 254 into a storage destination.

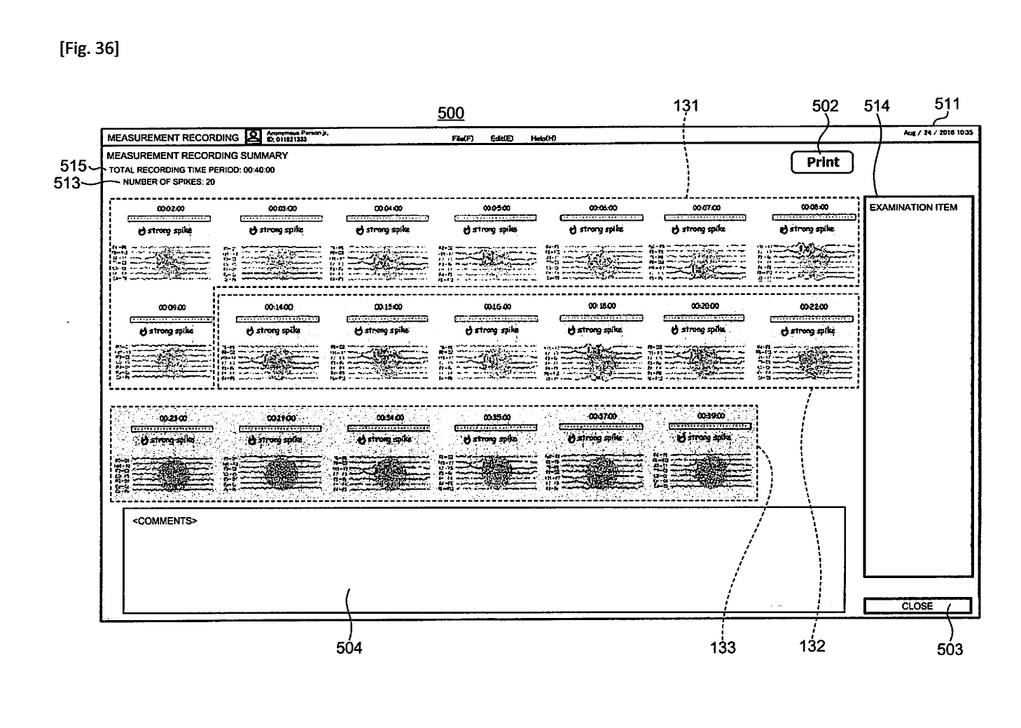

FIG. 36 is a sixth modification of the measurement recording result screen 500. The measurement recording result screen 500 illustrated in FIG. 36 is different from the examples described above in that it has a total recording time period 515, and strip waveforms are displayed in order of measurement files.

FIG. 36 illustrates an example where strip waveforms of the “first measurement file”, strip waveforms of the “second measurement file”, and strip waveforms of the “third measurement file” are displayed in this order. In FIG. 36, strip waveforms displayed in a rectangular frame 131, a rectangular frame 132, and a rectangular frame 133 correspond to the strip waveforms of the “first measurement file”, the strip waveforms of the “second measurement file”, and the strip waveforms of the “third measurement file”, respectively. As shaded in FIG. 36, each of the measurement files may have a different background color, so that the strip waveforms corresponding to each of the measurement files are able to be distinguished from the other. If distinction among the measurement files is unnecessary, the background colors may be not different from one another. Further, if the number of strip waveforms is large, by use of the scroll bar 505 illustrated in FIG. 32 or the like, the number of strip waveforms to be displayed at once on the screen 500 may be restricted.

According to this modification, since the number of annotations for the same subject, the number being necessary for analysis, is able to be checked for the whole measurement files, the advisability of remeasurement due to lack of the number of annotations is able to be checked easily.

### Seventh Modification of Measurement Recording Result Screen According to Fourth Embodiment: Display of Annotation Mark and Line

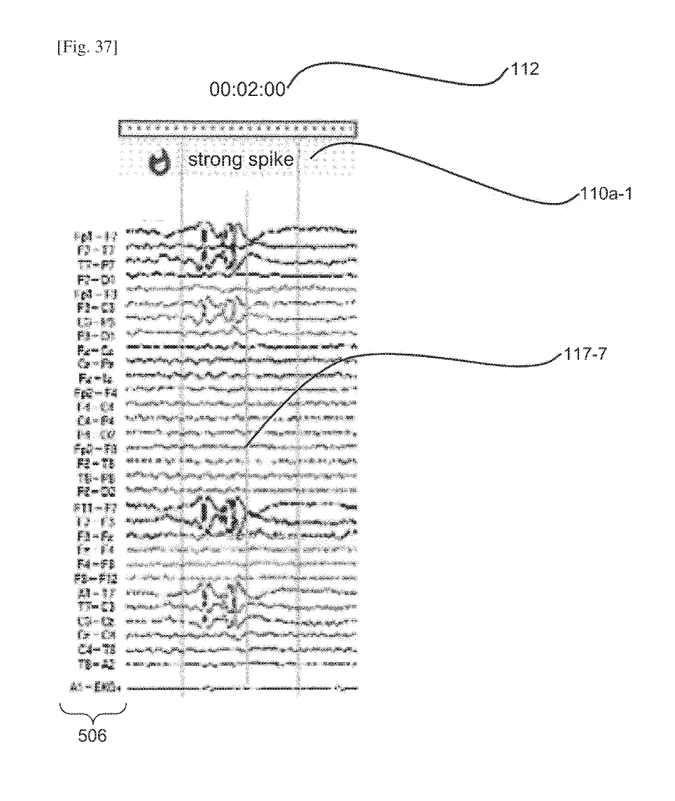

FIG. 37 illustrates a display mode in a case where the mark 103a-1 (see FIG. 31) and the attribute icon 106-1 (see FIG. 31) are not assigned. In this modification, as an example of emphasized display, the line 117-7 is displayed on a spot specified at the time of measurement recording, and the annotation 110a-1 indicating a result of the specification is displayed near the time axis 112. By not displaying the mark 103a-1 and the attribute icon 106-1, visibility of the waveforms at the background thereof is improved. The waveforms in this example are electroencephalography signal waveforms, but this modification is also applicable to magnetoencephalography signal waveforms.

### Eighth Modification of Measurement Recording Result Screen According to Fourth Embodiment: Superimposed Display of MEG with Reference to Zero

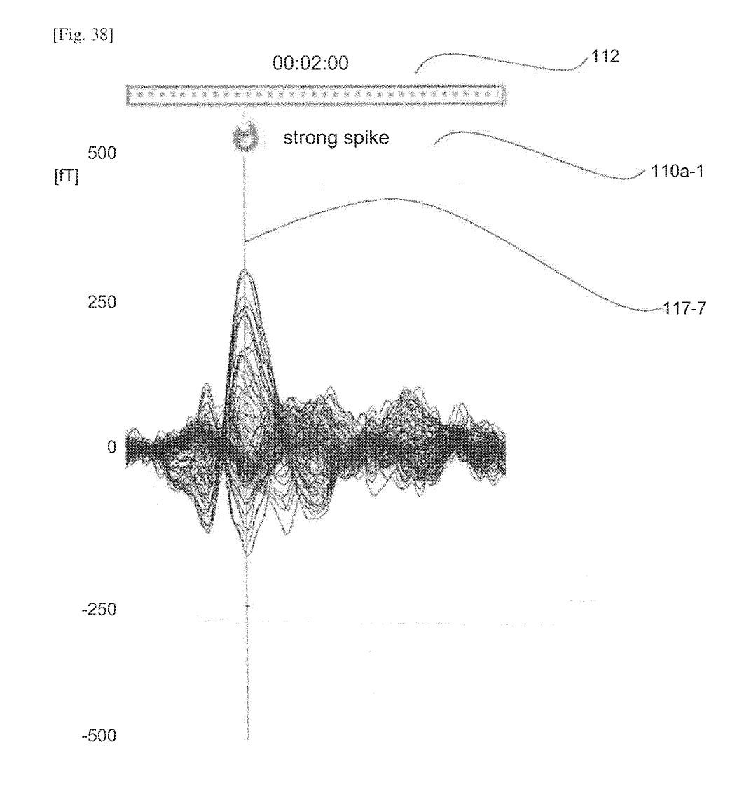

FIG. 38 illustrates a modification of the display method for waveforms of magnetoencephalography signals. In the above described embodiments, signal waveforms are displayed next to the sensor numbers (channel numbers) displayed as the channels 506. In this modification, waveforms of magnetoencephalography signals to be displayed are all displayed superimposed, with reference to zero. Thereby, visibility of waveforms that are comparatively large in amplitude is able to be improved. Further, for a layout where the waveforms of electroencephalography signals and magnetoencephalography signals are juxtaposed in the vertical direction on the screen 500, the vertical width of the waveforms of the magnetoencephalography signals is able to be decreased. Therefore, magnetoencephalographic waveforms and the electroencephalographic waveforms are able to be displayed largely in the vertical direction, and visibility thereof is improved. This modification is also applicable to the analysis screen 202.

The fourth embodiment is also applicable to the other embodiments. Further, any configuration described in the other embodiments but not described in the fourth embodiment may be applied to the fourth embodiment.

### Fifth Embodiment: Switch-Over from Summary Screen to Analysis Screen

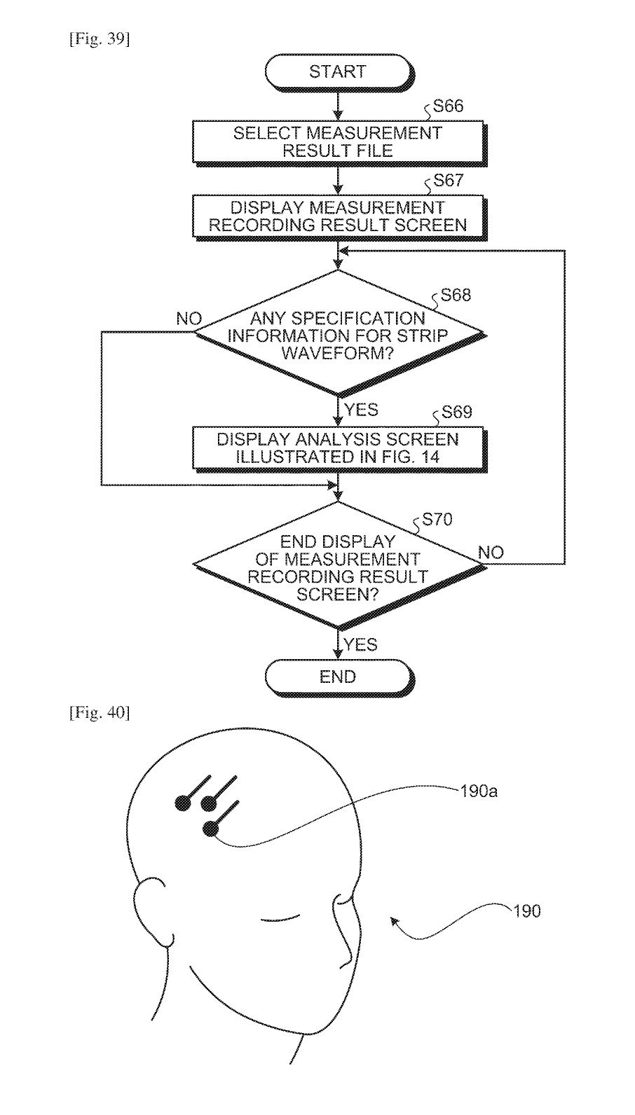

FIG. 39 is a flow chart illustrating an example of operation of switch-over from the measurement recording result screen 500 according to the fourth embodiment to the analysis screen 202 illustrated in FIG. 14. When the information processing apparatus 50 (control unit 250) receives selection of a measurement result file through a file list screen for measurement files (Step S66), the information processing apparatus 50 displays the measurement recording result screen 500 (Step S67).

Subsequently, when the measurer specifies (for example, double-clicks) a strip waveform desired to be displayed enlarged by using a mouse or the like and the information processing apparatus 50 (control unit 250) receives that operation (YES at Step S68), the information processing apparatus 50 displays the analysis screen 202 illustrated in FIG. 14 in a window different from that of the measurement recording result screen 500 (Step S69).

In the enlarged display area 200 illustrated in FIG. 14, enlarged waveforms of the strip waveform specified by the measurer on the measurement recording result screen 500 are displayed. Further, in a display area on the left side of the enlarged display area 200, waveforms of a time period around and including that of the specified strip waveform are displayed. Waveforms of a time width corresponding to the specified strip waveform are preferably displayed at the center of this display area.

Subsequently, the information processing apparatus 50 (control unit 250) determines whether to end the display of the measurement recording result screen 500 (Step S70). If there is another strip waveform desired to be enlarged (NO at Step S70), that strip waveform is specified through the measurement recording result screen 500 (Step S68), and the display of the analysis screen 202 is updated to waveforms and enlarged waveforms corresponding to the newly specified strip waveform (Step S69).

If the button 503 for closure of the window is pressed down on the measurement recording result screen 500 (NO at Step S68 and YES at Step S70), the measurement recording result screen 500 is closed and the flow is ended.

According to this modification, since a strip waveform is able to be enlarged and checked, whether the waveforms are usable on the analysis screen 202 is able to be determined more reliably.

### Sixth Embodiment

In each of the above described embodiments, tomographic images are described as an example of images to be displayed in the display window 190, but the images are not necessarily tomographic images. For example, the images may be spurious or schematic images or models, or animation images. Further, not being limited to two-dimensional images, the estimation results 190a may be displayed superimposed on, for example, a three-dimensional stereoscopic image illustrated in FIG. 40. In this case, if the stereoscopic image is rotated by an input device, such as a mouse, the measurer or the analyst is able to check the signal sources from a required angle.

The tomographic images, spurious or schematic images or models, animation images, and stereoscopic images are generally referred to, herein, as “biological images”.

Although the embodiments according to the present invention have been described thus far, the present invention is not limited to the above described embodiments as they are, and may be embodied by modification of the components upon implementation, without departing from the gist thereof. Further, by combination of plural components disclosed in the above described embodiments as appropriate, various inventions may be formed. For example, some components from all of the components described above in the embodiments may be omitted. Further, components from different embodiments and modifications may be combined as appropriate.

Further, a program executed by the above described biological signal measurement system 1 according to each of the embodiments: may be configured to be provided by being recorded on a computer readable recording medium, such as a CD-ROM, a flexible disk (FD), a CD-R, a digital versatile disk (DVD), or a universal serial bus (USB), in a file of an installable format or an executable format; or may be configured to be provided or distributed via a network, such as the Internet. Further, various programs may be configured to be provided by being incorporated in a ROM or the like beforehand.

According to an embodiment, reliability of identification of a target part being a cause of a case is able to be improved.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

### REFERENCE SIGNS LIST

1 Biological signal measurement system
3 Measurement apparatus
21 CPU
22 RAM
23 ROM
24 Auxiliary storage device
25 Input-output interface
27 Bus
28 Display device
40 Server
50 Information processing apparatus
250 Control unit
251 Display control unit
252 Analysis unit
253 Sensor information acquisition unit
254 Recording/analysis information storage unit
255 Operation information input unit
256 Print data output unit
400 Screen
500 Measurement recording result screen

What is claimed is:

1. An information processing apparatus comprising:
a display control unit configured to execute control to display a plurality of partial waveform data in parallel in a time axis direction of the plurality of partial waveform data, the plurality of partial waveform data each representing temporal change of one or more biological signals and each respectively representing biological waveform data corresponding to a respective partial time period, spots from among the biological waveform data being specifiable, the display control unit being further configured to execute control to display the spots of the plurality of partial waveform data, with emphasis, upon being specified.

2. The information processing apparatus according to claim 1, wherein the control to display with emphasis is executed to display a line extending in a direction intersecting a measurement time direction of a waveform representing the biological waveform data.

3. The information processing apparatus according to claim 1, wherein
the one or more biological signals are biological signals acquired by a plurality of measurement methods, and
the display control unit is configured to execute control to display at least one or more of the one or more biological signals acquired by the plurality of measurement methods.

4. The information processing apparatus according to claim 3, wherein the plurality of measurement methods comprise MEG and EEG.

5. The information processing apparatus according to claim 1, wherein the display control unit is configured to:
execute, for a first display area, the control to display in parallel and the control to display with emphasis; and
execute, for a second display area, control to display a biological image including a signal source superimposed on the biological image, the signal source corresponding to one of the plurality of partial waveform data.

6. The information processing apparatus according to claim 5, wherein the display control unit is configured to three-dimensionally display the biological image.

7. The information processing apparatus according to claim 5, wherein the display control unit is configured to, upon one of the plurality of partial waveform data being selected, display the biological image including a signal source corresponding to the one of the plurality of partial waveform data selected, and display information including the signal source superimposed on the biological image.

8. The information processing apparatus according to claim 7, wherein the display control unit is configured to display the signal source corresponding to the one of the plurality of partial waveform data selected and superimposed on the biological image, in a display mode different from another signal source.

9. The information processing apparatus according to claim 1, wherein the display control unit is configured to display, side by side, the plurality of partial waveform data, from partial waveform data wherein a measurement was previously performed, toward partial waveform data wherein a measurement is later performed.

10. The information processing apparatus according to claim 7, wherein the display control unit is configured to execute control to display a magnetoencephalographic distribution diagram, upon one of the plurality of partial waveform data being selected, based on the one of the plurality of partial waveform data selected.

11. The information processing apparatus according to claim 1, wherein the display control unit is configured to change a display color, upon one of the plurality of partial waveform data being selected, of the one of the plurality of partial waveform data selected.

12. The information processing apparatus according to claim 1, wherein the display control unit is configured to display a time axis corresponding to the partial waveform data.

13. The information processing apparatus according to claim 12, wherein the display control unit is configured to display an annotation near the partial waveform data or near the time axis.

14. The information processing apparatus according to claim 1, wherein the display control unit is configured to display a time axis for an overall biological waveform data, and execute display such that a plurality of the spots are recognizable on the time axis when selected.
